SEQUENCE LISTING (6 sequences)

SEQ ID NO: 1 - protein, 1090 aa, Homo sapien
MAAGCLLALTLTLFQSLLIGPSSEEPFPSAVTIKSWVDKMQEDLVTLAKTASGVNQLVDIYEKYQDLYTVEPNNARQLVEIAARDIEKLLSNRSKALVSLALEAEKVQAAHQWREDFASNEVVYYNAKDDLDPEKNDSEPGSQRIKPVFIEDANFGRQISYQHAAVHIPTDIYEGSTIVLNELNWTSALDEVFKKNREEDPSLLWQVFGSATGLARYYPASPWVDNSRTPNKIDLYDVRRRPWYIQGAASPKDMLILVDVSGSVSGLTLKLIRTSVSEMLETLSDDDFVNVASFNSNAQDVSCFQHLVQANVRNKKVLKDAVNNITAKGITDYKKGFSFAFEQLLNYNVSRANCNKIIMLFTDGGEERAQEIFNKYNKDKKVRVFRFSVGQHNYERGPIQWMACENKGYYYEIPSIGAIRINTQEYLDVLGRPMVLAGDKAKQVQWTNVYLDALELGLVITGTLPVFNITGQFENKTNLKNQLILGVMGVDVSLEDIKRLTPRFTLCPNGYYFAIDPNGYVLLHPNLQPKNPKSQEPVTLDFLDAELENDIKVEIRNKMIDGESGEKTFRTLVKSQDERYIDKGNRTYTWTPVNGTDYSLLVLPTYSFYYIKAKLEETITQARSKKGKMKDSETLKPDNFEESGYTFIAPRDYCNDLKISDNNTEFLLNFNEFIDRKTPNNPSCNADLINRVLLDAGFTNELVQNYWSKQKNIKGVKARFVVTDGGITRVYPKEAGENWQENPETYEDSFYKRSLDNDNYVFTAPYFNKSGPGAYESGIMVSKAVEIYIQGKLLKPAVVGIKIDVNSWIENFTKTSIRDPCAGPVCDCKRNSDVMDCVILDDGGFLLMANHDDYTNQIGRFFGEIDPSLMRHLVNISVYAFNKSYDYQSVCEPGAAPKQGAGHRSAYVPSVADILQIGWWATAAAWSILQQFLLSLTFPRLLEAVEMEDDDFTASLSKQSCITEQTQYFFDNDSKSFSGVLDCGNCSRIFHGEKLMNTNLIFIMVESKGTCPCDTRLLIQAEQTSDGPNPCDMVKQPRYRKGPDVCFDNNVLEDYTDCGGVSGLNPSLWYIIGIQFLLLWLVSGSTHRLL

SEQ ID NO: 2 - protein, 1150 aa, Homo sapien [sequence omitted]

SEQ ID NO: 3 - protein, 1090 aa, Homo sapien
MAGPGSPRRASRGASALLAAALLYAALGDVVRSEQQIPLSVVKLWASAFGGEIKSIAAKYSGSQLLQKKYKEYEKDVAIEEIDGLQLVKKLAKNMEEMFHKKSEAVRRLVEAAEEAHLKHEFDADLQYEYFNAVLINERDKDGNFLELGKEFILAPNDHFNNLPVNISLSDVQVPTNMYNKDPAIVNGVYWSESLNKVFVDNFDRDPSLIWQYFGSAKGFFRQYPGIKWEPDENGVIAFDCRNRKWYIQAATSPKDVVILVDVSGSMKGLRLTIAKQTVSSILDTLGDDDFFNIIAYNEELHYVEPCLNGTLVQADRTNKEHFREHLDKLFAKGIGMLDIALNEAFNILSDFNHTGQGSISQAIMLITDGAVDTYDTIFAKYNWPDRKVRIFTYLIGREAAFADNLKWMACANKGFFTQISTLADVQENVMEYLHVLSRPKVIDQEHDVVWTEAYIDSTLPQAQKLTDDQGPVLMTTVAMPVFSKQNETRSKGILLGVVGTDVPVKELLKTIPKYKLGIHGYAFAITNNGYILTHPELRLLYEEGKKRRKPNYSSVDLSEVEWEDRDDVLRNAMVNRKTGKFSMEVKKTVDKGKRVLVMTNDYYYTDIKGTPFSLGVALSRGHGKYFFRGNVTIEEGLHDLEHPDVSLADEWSYCNTDLHPEHRHLSQLEAIKLYLKGKEPLLQCDKELIQEVLFDAVVSAPIEAYWTSLALNKSENSDKGVEVAFLGTRTGLSRINLFVGAEQLTNQDFLKAGDKENIFNADHFPLWYRRAAEQIPGSFVYSIPFSTGPVNKSNVVTASTSIQLLDERKSPVVAAVGIQMKLEFFQRKFWTASRQCASLDGKCSISCDDETVNCYLIDNNGFILVSEDYTQTGDFFGEIEGAVMNKLLTMGSFKRITLYDYQAMCRANKESSDGAHGLLDPYNAFLSAVKWIMTELVLFLVEFNLCSWWHSDMTAKAQKLKQTLEPCDTEYPAFVSERTIKETTGNIACEDCSKSFVIQQIPSSNLFMVVVDSSCLCESVAPITMAPIEIRYNESLKCERLKAQKIRRRPESCHGFHPEENARECGGAPSLQAQTVLLLLPLLLMLFSR

SEQ ID NO: 4 - protein, 1137 aa, Homo sapien
MVCGCSALLPLPNPRPTMPATPNFLANPSSSSRWIPLQPMPVAWAFVQKTSALLWLLLLGTSLSPAWGQAKIPLETVKLWADTFGGDLYNTVTKYSGSLLLQKKYKDVESSLKIEEVDGLELVRKFSEDMENMLRRKVEAVQNLVEAAEEADLNHEFNESLVFDYYNSVLINERDEKGNFVELGAEFLLESNAHFSNLPVNTSISSVQLPTNVYNKDPDILNGVYMSEALNAVFVENFQRDPTLTWQYFGSATGFFRIYPGIKWTPDENGVITFDCRNRGWYIQAATSPKDIVILVDVSGSMKGLRMTIAKHTITTILDTLGENDFVNIIAYNDYVHYIEPCFKGILVQADRDNREHFKLLVEELMVKGVGVVDQALREAFQILKQFQEAKQGSLCNQAIMLISDGAVEDYEPVFEKYNWPDCKVRVFTYLIGREVSFADRMKWIACNNKGYYTQISTLADTQENVMEYLHVLSRPMVINHDHDIIWTEAYMDSKLLSSQAQSLTLLTTVAMPVFSKKNETRSHGILLGVVGSDVALRELMKLAPRYKLGVHGYAFLNTNNGYILSHPDLRPLYREGKKLKPKPNYNSVDLSEVEWEDQAESLRTAMINRETGTLSMDVKVPMDKGKRVLFLTNDYFFTDISDTPFSLGVVLSRGHGEYILLGNTSVEEGLHDLLHPDLALAGDWIYCITDIDPDHRKLSQLEAMIRFLARKDPDLECDEELVREVLFDAVVTAPMEAYWTALALNMSEESEHVVDMAFLGTRAGLLRSSLFVGSEKVSDRKFLTPEDEASVFTLDRFPLWYRQASEHPAGSFVFNLRWAEGPESAGEPMVVTASTAVAVTVDKRTAIAAAAGVQMKLEFLQRKFWAATRQCSTVDGPCTQSCEDSDLDCFVIDNNGFILISKRSRETGRFLGEVDGAVLTQLLSMGVFSQVTMYDYQAMCKPSSHHHSAAQPLVSPISAFLTATRWLLQELVLFLLEWSVWGSWYDRGAEAKSVFHHSHKHKKQDPLQPCDTEYPVFVYQPAIREANGIVECEPCQKVFVVQQIPNSNLLLLVTDPTCDCSIFPPVLQEATEVKYNASVKCDRMRSQKLRRRPDSCHAFHPEENAQDCGGASDTSASPPLLLLPVCAWGLLPQLLR

SEQ ID NO: 5 - protein, 48 aa, Artificial Sequence
  Synthetic Constuct
RIVRVFTFSVGQHNYDVTPLQWMACANKGYYFEIPSIGAIRINTQEKK

SEQ ID NO: 6 - protein, 46 aa, Artificial Sequence
  Synthetic Constuct
KKVRVRFSVGQHNYERGPIQWMACENKGYYYEIPSIGAIRINTQER

(19) United States
(12) Patent Application Publication
(10) Pub. No.: US 2011/0287014 A1
(43) Pub. Date: Nov. 24, 2011

(54) ANTIBODIES DIRECTED AGAINST CALCIUM CHANNEL SUBUNIT ALPHA2/DELTA AND METHODS USING SAME

(76) Inventor: Arnon Rosenthal, Woodside, CA (US)

(21) Appl. No.: 13/130,941

(22) PCT Filed: Nov. 24, 2009
(86) PCT No.: PCT/US09/65782

§ 371 Date: Aug. 8, 2011

(30) Foreign Application Priority Data
Nov. 24, 2008 (US) 61/117514

(51) Int. Cl. A61K 39/395; C07H 21/00; C12N 15/63; C12N 5/10; A61P 25/16; C12N 1/19; C12P 21/00; A61P 25/00; A61P 25/28; C07K 16/36; C12N 1/21

(52) U.S. Cl. 424/139.1; 530/387.9; 530/387.3; 536/23.53; 435/320.1; 435/331; 435/252.33; 435/252.31; 435/254.21; 435/254.2; 435/69.6

(57) ABSTRACT

The present invention provides compositions comprising antibodies or polypeptides that specifically bind to an epitope in a von Willebrand Factor A (VWF-A) domain of a calcium channel subunit α2δ and induces synaptogenesis and methods of using the compositions for inducing synaptogenesis and neurite outgrowth.

### RELATED APPLICATIONS

This application claims the priority benefit of U.S. provisional application U.S. Ser. No. 61/117,514, filed Nov. 24, 2008., which is incorporated herein by reference in its entirety.

### TECHNICAL FIELD

The present invention relates generally to compositions and methods for inducing synaptogenesis and axon and dendritic growth. Specifically, the invention includes use of novel antibodies or polypeptides that specifically bind to an epitope in a Von Willebrand Factor (VWF-A) domain of a calcium channel subunit α2δ for inducing synaptogenesis.

### BACKGROUND OF THE INVENTION

Synapses are specialized junctions through which the cells of the nervous system communicate. Their establishment requires an interaction between the axons and the dendrites, accompanied by the organization of many pre- and post-synaptic specializations (Davis, 2000; Garner, 2006; Kennedy, 2006; Ziv, 2004). Several neuronal cell-surface molecules and secreted signals have been shown to be involved in processes that lead to synaptic organization and maturation (Chih, 2005; Nishimune, 2004; Scheiffele, 2003; Umemori, 2004; Washbourne, 2004), but the nature of the extracellular and intracellular mechanisms that induce the initial formation of synaptic adhesions remain poorly understood. Accumulating evidence has shown that astrocytes, the most abundant cell type in the brain, play active roles in the formation and function of synapses via the secretion of Thrombospondins (TSP), a family of 5 secreted proteins which are necessary and sufficient synaptogenic signal (Christopherson, 2005). TSP2 is present in astrocyte-conditioned media (ACM), and is responsible for the ability of astrocytes to increase synapse number in culture (Christopherson, 2005). TSP1 and TSP2 are expressed in the brain during early postnatal stages when the majority of synapses are forming, and, when both proteins are absent from the adult brain, the amount of synaptogenesis is significantly reduced (Christopherson, 2005). TSP is able to trigger the formation of both pre- and post-synaptic specializations, and also align both sides to oppose each other, indicating that it is able to promote synaptic adhesion and initiate the events that lead to the structural establishment of the synapse. Interestingly, these TSP-induced synapses are ultrastructurally identical to fully developed synapses (determined by electron microscopy) and are presynaptically active (determined by FM-dye uptake assay), however they are postsynaptically silent due to the lack of surface AMPA receptors (Christopherson, 2005).

Recently, the calcium channel subunit α2δ-1 has been identified as the TSP receptor involved in synapse formation (Susman, 2007 society for neuroscience abstract 571.2/G52; Eroglu, 2009). TSP and 0261 may interact with neuroligin 1 to enhance synaptogenesis (Xu, 2009). The calcium channel subunits alpha-2/delta constitutes a family of 4 structurally related proteins that are thought to act as subunits in voltage dependent calcium channels e.g. (Klugbauer, 2003). Calcium channels mediate the influx of calcium ions into the cell upon membrane polarization and consist of a complex of alpha-1, alpha-2/delta, beta, and gamma subunits in a 1:1:1:1 ratio. The first member of the alpha-2/delta1 was isolated as a structural component of the dihydropyridine binding L-type calcium channel complex from skeletal muscle (Arikkath, 2003). The alpha-2/delta family members are selectively expressed in different neuronal and non neuronal tissues (Cole, 2005). For example alpha-2/delta2 is expressed primarily in GABAergic neurons (Cole, 2005). Interestingly a sporadic mutation in mice, which disrupts the alpha-2/delta4 gene, leads to a severe loss of ribbon synapses in the photoreceptor cells (Wyciskl, 2006). In addition, mutations in alpha-2/delta3 cause defects in synaptic transmission and a morphological defect in presynaptic organization at the Drosophila neuromuscular junction (Dickman, 2008) suggesting that multiple members of the alpha-2/delta members control synaptogenesis. In addition other alpha-2/delta isoforms may exist (Whittaker, 2002). Each of these alpha-2/delta isoforms shares a large extracellular domain and thus it is possible that binding of TSP or other natural or manmade ligands to these domains initiates synaptogenesis and/or helps to control the formation of specific synaptic connections. Alpha-2/delta members are post-translationally cleaved into alpha-2 and delta components that remain associated via disulfide bridges. The alpha-2 portion of the protein (around 950 amino acids) is entirely extracellular while the delta portion has a small extracellular part that is attached to alpha-2, and a transmembrane domain with a very short cytoplasmic tail that tethers the whole molecule to the membrane (Davies, 2007). The alpha-2/delta1 and 2 are thought to be the targets for the anti-epileptic and anti-pain drug gabapentin (e.g. Neurontin and Lyrica) (Field, 2006; Gee, 1996).

Alpha-2/delta1 has been shown to have three known roles in regulating L-type voltage-dependent calcium channel function (Arikkath, 2003). It modulates the voltage dependent behavior and inactivation kinetics of L-type calcium channels. Overexpression of alpha-2/delta1 with pore forming calcium channel subunit alpha1 also significantly enhances calcium currents by increasing the number of alpha1 subunits present on the cell surface (Arikkath, 2003). Although much research on alpha-2/delta1 has focused on its role in the regulation of calcium channel function and trafficking, the presence of a large extracellular region containing a well known protein-protein interaction fold, the Von Willebrand Factor A (VWF-A) domain, suggests that this protein could potentially serve as a receptor for extracellular ligands.

The von Willebrand factor is a large multimeric glycoprotein found in blood plasma. Mutant forms are involved in the aetiology of bleeding disorders (Ruggeri, 1993). In von Willebrand factor, the type A domain (vWF) is the prototype for a protein superfamily. The vWF domain is found in various plasma proteins: complement factors B, C2, CR3 and CR4; the integrins (1-domains); collagen types VI, VII, XII and XIV; and other extracellular proteins (Colombatti, 1993; Perkins, 1994; Bork, 1999) Proteins that incorporate vWF domains participate in numerous biological events (e.g., cell adhesion, migration, homing, pattern formation, and signal transduction), involving interaction with a large array of ligands often via metal ion-dependent adhesion sites (MIDAS) (Colombatti, 1993).

Secondary structure prediction from 75 aligned vWF sequences has revealed a largely alternating sequence of alpha-helices and beta-strands (Perkins, 1994). Fold recognition algorithms were used to score sequence compatibility with a library of known structures: the vWF domain fold was predicted to be a doubly-wound, open, twisted beta-sheet flanked by alpha-helices (Edwards, 1995).

3D structures have been determined for the I-domains of integrins CD11b (with bound magnesium) and CD11a (with bound manganese) (Qu, 1995). The domain adopts a classic alpha/beta Rossmann fold and contains an unusual metal ion coordination site at its surface. It has been suggested that this site represents a general metal ion-dependent adhesion site (MIDAS) for binding protein ligands (Lee, 1995). The residues constituting the MIDAS motif in the CD11b and CD11a I-domains are completely conserved, but the manner in which the metal ion is coordinated differs slightly (Qu, 1995).

VWF-A domain is a 3-element fingerprint that provides a signature for the vWF domain superfamily. The fingerprint was derived from an initial alignment of 14 sequences. Motif 1 includes the first beta-strand and 3 conserved residues involved in metal ion coordination in I-domains (Asp and 2 serines in positions 8, 10 and/or 12, respectively); motif 2 spans strands beta-2 and beta-T; and motif 3 encodes beta-strand 3 and a conserved Asp (in position 7), which coordinates the metal ion (Lee, 1995; Qu, 1995). Three iterations on OWL27.0 were required to reach convergence, at which point a true set comprising 56 sequences was identified. Numerous partial matches were also found. VWF-A domains are well documented to be protein-protein interaction domains, and act as conformational switches that alter a protein's structure upon binding to its ligand (Bork, 1991; Whittaker, 2002).

Although antibodies against the VWF-A domain have been previously described, these were directed against the VWF-A domain of the human vWF and to inhibits the binding of vWF to platelets; U.S. Pat. No. 6,251,393—Conformation-specific anti-von Willebrand Factor antibodies. Likewise, antibodies have been described to the VWF-A like-domain of the integrin LFA-1 (L2, CD11a/CD18) which regulates adhesive functions and migration of lymphocytes and most other leukocytes. This domain is structurally similar to the von Willebrand factor A domain/dinucleotide-binding fold and is thought to participate in the binding to ICAM-1 (Huang, 1995a&b; Huang, 2000; Landis, 1993). The antibodies were shown to facilitate the interaction between LFA-1 and ICAM-1 but were not shown to elicit any biological function. There are also reports on antibodies against three different regions of the beta-1 integrin which induced adherence of the T-leukemic cell line Jurkat to collagen and fibronectin. Some of these antibodies bind the alpha-A domain of beta-1 integrin which a similar structure to the A-domains found in the protein von Willebrand factor. (Wilkins, 1995). Antibodies against the human alpha-2/delta1 (Taylor, 2008), alpha-2/delta2 and Human alpha-2/delta3 (Marais, 2001) have been reported, but these antibodies did not show any biological activity.

Lose of synapses is the hallmark of brain injury and neurodegenerative diseases. For example, synapse loss in frontal cortex biopsies of Alzheimer's disease patients correlates with the severity of cognitive decline (DeKosky, 1990). Likewise, early and selective loss of neuromuscular synapse is associated with motoneuron diseases (Frey, 2000; Sasaki, 1994). Conversely, inhibiting axon degeneration and synapse loss attenuates apoptosis and disease progression motoneuron diseases (Ferri, 2003). Early lose of synapse is also observed following spinal cord injury (Burns, 2007), in a primate model of Parkinson's disease (Hen, 2008), progressive supranuclear palsy (Bigio, 2001) and Tauopathy (Yoshiyama, 2007). In addition, mechanisms that increase synapse elimination were shown to be up regulated in glaucoma, Alzheimer's disease and cerebral ischemia (e.g. Stevens, 2007; Mack, 2006), while deficit in the ability to induce synapses was associated with poor recovery from stroke (Liauw, 2008).

Specialized nerve endings in organs such as the eye, ear, tongue, nose, or skin, where sensory neurons are concentrated and function as a sensors are thought to be primitive synapses and their function was shown to be regulated by TSP like proteins (Bacaj, 2008) which are ligands to the alpha-2 and delta proteins. The alpha-2/delta ligand TSP was also shown to induce neurite outgrowth (Arber, 1995).

All references, publications, and patent applications disclosed herein are hereby incorporated by reference in their entirety.

### SUMMARY OF THE INVENTION

The present invention provides isolated antibodies and polypeptides (which may or may not be an antibody) that specifically bind to an epitope in a von Willebrand Factor A (VWF-A) domain of a calcium channel subunit selected from the group consisting of α2δ1, α2δ2, α2δ3, and α2δ4 and induces synaptogenesis of a neuronal cell expressing the calcium channel subunit.

In some embodiments, the antibody or the polypeptide binds to an epitope within amino acids of about 253 to about 430 of human α2δ1 shown in SEQ ID NO:1. In some embodiments, the antibody binds to an epitope within amino acids of about 386 to about 425 of human α2δ1 shown in SEQ ID NO:1. In some embodiments, the antibody or the polypeptide binds to a peptide having the amino acid sequence of SEQ ID NO:6 (MZp110). In some embodiments, the antibody or the polypeptide binds to an epitope within amino acids of about 291 to about 469 of human α2δ2 isoform shown in SEQ ID NO:2. In some embodiments, the antibody binds to an epitope within amino acids of about 421 to about 464 of human α2δ3 shown in SEQ ID NO:3. In some embodiments, the antibody or the polypeptide binds to a peptide having the amino acid sequence of SEQ ID NO:5 (MZp109).

In some embodiments, the antibody binds to an epitope within amino acids of about 256 to about 438 of human α2δ3 shown in SEQ ID NO:3. In other embodiments, the antibody binds to an epitope within amino acids of about 291 to about 473 of human α2δ4 shown in SEQ ID NO:4.

In some embodiments, the antibody is a monoclonal antibody. In some embodiments, the antibody is a human antibody, a humanized antibody, or a chimeric antibody.

In some embodiments, the antibody is antibody 5A5 that is produced by the host cell line MZP109-5A5 deposited on ______ at the American Type Culture Collection with patent deposit designation of ______. Antibody 5A5 binds to both MZp109 and Mzp110. In some embodiments, the antibody comprises a fragment or a region of antibody 5A5. In some embodiments, the antibody comprises the heavy chain variable region and/or the light chain variable region of antibody 5A5. In some embodiments, the antibody is a chimeric antibody comprising the heavy chain variable region and/or the light chain variable region of antibody 5A5 and constant regions derived from constant regions of a heavy chain and a light chain of one or more human antibodies. In some embodiments, the antibody comprises one, two, or three of the CDRs from the heavy chain of antibody 5A5, and/or one, two, or three of the CDRs from the light chain of antibody 5A5. In some embodiments, the antibody is a humanized antibody comprising the three CDRs from the heavy chain of antibody 5A5, and/or the three CDRs from the light chain of antibody 5A5.

In some embodiments, the antibody is antibody 3B4 that is produced by the host cell line MZP110-3B4 deposited on ______ at the American Type Culture Collection with patent deposit designation of ______. Antibody 3B4 binds to both MZp109 and Mzp110. In some embodiments, the antibody comprises a fragment or a region of antibody 3B4. In some embodiments, the antibody comprises the heavy chain variable region and/or the light chain variable region of antibody 3B4. In some embodiments, the antibody is a chimeric antibody comprising the heavy chain variable region and/or the light chain variable region of antibody 3B4 and constant regions derived from constant regions of a heavy chain and a light chain of one or more human antibodies. In some embodiments, the antibody comprises one, two, or three of the CDRs from the heavy chain of antibody 3B4, and/or one, two, or three of the CDRs from the light chain of antibody 3B4. In some embodiments, the antibody is a humanized antibody comprising the three CDRs from the heavy chain of antibody 3B4, and/or the three CDRs from the light chain of antibody 3B4.

The invention also provides a composition comprising one or more of the antibodies or polypeptides described herein. In some embodiments, the composition further comprises a pharmaceutically acceptable carrier.

The invention also provides an isolated polynucleotide comprising a nucleotide sequence that encodes any of the antibodies or polypeptides described herein. The invention also provides vectors (including expression vectors) comprising a polynucleotide described herein. The invention provides host cells comprising a polynucleotide described herein. In some embodiments, the host cell is a mammalian cell (such as a CHO cell, a mouse hybridoma cell line), a yeast cell, a plant cell, an insect cell. The invention also provides methods of producing an antibody or a polypeptide described herein, the method comprising culturing a host cell that produces the antibody or the polypeptide, and recovering the antibody or the polypeptide from the cell culture.

The invention provides methods for screening an antibody or a polypeptide for activity in inducing synaptogenesis, the methods comprising 1) contacting neuronal cells expressing a calcium channel subunit selected from the group consisting of α2δ1, α2δ2, α2δ3, and α2δ4 with an candidate antibody or polypeptide that specifically binds to an epitope in a von Willebrand Factor A (VWF-A) domain of the calcium channel subunit; and 2) quantitating formation of synapses in the cell culture in the presence of the candidate antibody or polypeptide.

The invention provides methods for inducing synaptogenesis in an individual comprising administering to the individual in need of synaptogenesis an effective dose of an antibody or a polypeptide that specifically binds to an epitope in a von Willebrand Factor A (VWF-A) domain of a calcium channel subunit selected from the group consisting of α2δ1, α2δ2, α2δ3, and α2δ4 and induces synaptogenesis. Any of the antibodies or polypeptides described herein may be used.

The invention provides methods for inducing neurite outgrowth in an individual comprising administering to the individual in need of neurite outgrowth an effective dose of an antibody or a polypeptide that specifically binds to an epitope in a von Willebrand Factor A (VWF-A) domain of a calcium channel subunit selected from the group consisting of α2δ1, α2δ2, α2δ3, and α2δ4 and induces neurite outgrowth. Any of the antibodies or polypeptides described herein may be used.

In some embodiments, the individual has suffered synapse loss as a result of senescence or normal aging. In some embodiments, the individual has suffered synapse loss as a result of Alzheimer's disease, Parkinson's disease, ALS, multiple sclerosis, Huntington disease, Down syndrome, spinal mascular atrophy, stroke, spinal cord injury, myofiber atrophy, denervation atrophy, or glaucoma. In some embodiments, the individual has suffered a macular degeneration, a hearing loss, a diabetic neuropathy, or a chemotherapy induced neuropathy. In some embodiments, the individual has suffered synapse loss as a result of a psychiatric disorder selected from the group consisting of depression, schizophrenia, autism, and aggression. In some embodiments, the individual has suffered synapse loss as a result of a viral infection selected from the group consisting of poliomyelitis, West Nile virus, or human immunodeficiency virus (HIV). In some embodiments, the individual has suffered synapse loss as a result of a Prion disease. In some embodiments, the Prion disease is a Creutzfeldt-Jakob disease.

In some embodiments, synaptogenesis is increased at a neuromuscular junction. In some embodiments, synaptogenesis is increased in the brain, spinal cord or sense organs. In some embodiments, the function of sense organs is enhanced.

The invention also provides methods for treating an individual suffering from a disease associated with loss of synapses or preventing an individual from adverse effects of deficits in synaptogenesis comprising administering to the individual an effective dose of an antibody or a polypeptide described herein. The disease includes, but is not limited to, Alzheimer's disease, Parkinson's disease, ALS, multiple sclerosis, Huntington disease, Down syndrome, spinal mascular atrophy, stroke, spinal cord injury, myofiber atrophy, denervation atrophy, glaucoma, macular degeneration, hearing loss, diabetic neuropathy, neuropathy induced by chemotherapy, depression, schizophrenia, autism, aggression, viral infection selected from the group consisting of poliomyelitis, West Nile virus, or human immunodeficiency virus (HIV), and Prion disease (e.g., Creutzfeldt-Jakob disease).

The invention also provides articles of manufacture and kits comprising an antibody or a polypeptide described herein. The kits may further comprise instructions for use in any of the methods described herein.

It is to be understood that one, some, or all of the properties of the various embodiments described herein may be combined to form other embodiments of the present invention.

### BRIEF DESCRIPTION OF THE DRAWINGS

FIG. 1 shows the amino acid sequence of human voltage-dependent calcium channel subunit α2δ1 (SEQ ID NO: 1) (Gene name: CACNA2D1; Synonyms: CACNL2A, CCHL2A, and MHS3; Entry name: CA2D1_HUMAN; Primary accession number: P54289). VWF-A domain is underlined.

FIG. 2 shows the amino acid sequence of a human voltage-dependent calcium channel subunit α2δ2 (SEQ ID NO:2) (Gene name: CACNA2D2; Synonym:KIAA0558; entry name: CA2D2_HUMAN; Identifier: Q9NY47). VWF-A domain is underlined. There are five isoforms of α2δ2 produced as result of alternative splicing.

FIG. 3 shows the amino acid sequence of human voltage-dependent calcium channel subunit α2δ3 (SEQ ID NO:3) (Gene name: CACNA2D3; Entry name. CA2D3_HUMAN; Identifier: Q8IZS8). VWF-A domain is underlined.

FIG. 4 shows the amino acid sequence of human voltage-dependent calcium channel subunit α2δ4 (SEQ ID NO:4) (Gene name: CACNA2D4; Entry name: CA2D4_HUMAN; Identifier: Q7Z3S7). VWF-A domain is underlined.

FIG. 5: (A) Western blot analysis shows specific binding of 5A5 and 3B4 antibodies to human embryonic kidney cell membrane (HEK293) that over-expresses the rat α2δ1 protein. (B) Fluorescent immunostaining of cell surface of HEK293 cells overexpressing the rat α2δ1 with antibodies 5A5 and 3A4. (C) Analysis of the synaptic inducing activity of antibodies 5A5 and 3B4. Retinal ganglion neurons (RGC) were purified with greater than 99.5% purity from P5 Sprague-Dawley rats. RGC were cultured in serum free media with these antibodies in the presence or absence of TSP and synapse number was analyzed. Both 5A5 and 3B4 were found to induce synapse formation in the absence of TSP while a control antibody (OX7) against another RGC surface receptor Thy1 did not. The synaptogenic effect of 5A5 and 3B4 was not changed in the presence of TSP.

### DETAILED DESCRIPTION OF THE INVENTION

The present invention is based on the discovery that antibodies directed against a VWF-A domain of calcium channel subunit α2δ can activate α2δ to induce synapse formation. Compositions comprising the antibodies or polypeptides against a VWF-A domain of calcium channel subunit α2δ and methods of using the compositions for inducing synaptogenesis are provided. Synaptogenesis is enhanced by contacting neurons with antibodies or polypeptides that specifically bind to an epitope in a VWF-A domain of any member of the calcium channel subunit α2δ protein family. The methods and compositions may be used for protecting or treating an individual suffering from or preventing an individual from adverse effects of deficits in synaptogenesis. These findings have broad implications for a variety of clinical conditions, including traumatic brain injury, neurodegerative diseases, and other conditions where synapses fail to form, form inappropriately or are lost.

The antibody specific for an epitope of a VWF-A domain of a calcium channel subunit α2δ1, α2δ2, α2δ3, or α2δ4 is useful in stimulating synapse formation in areas where synapses or nerve endings in sense organs are lost or become dysfunctional due to neurodegenerative diseases, e.g. Alzheimer's disease, Parkinson's disease, amyotrophic lateral sclerosis (ALS), multiple sclerosis, Huntington disease, Down syndrome, spinal mascular atrophy, stroke, spinal cord injury, myofiber atrophy, denervation atrophy, glaucoma, macular degeneration, hearing loss, diabetic neuropathy, chemotherapy induced neuropathy, psychiatric disorders (e.g. depression, schizophrenia, autism, aggression), viral infection (e.g. poliomyelitis, West Nile virus, and HIV), Prion disease (e.g. Creutzfeldt-Jakob disease), stroke, neuropathy aging, etc. The synaptogenesis antibodies or polypeptides described herein may be administered by any methods including topically, e.g. to optic nerves or spinal cord.

### DEFINITIONS

Synapses, as used herein, refer to either specialized junctions through which the cells of the nervous system communicate with each other or with other organs or tissue, including specialized nerve endings that act as sensors in sense organs.

Synaptogenesis, as used herein, refers to the process by which pre- and/or post-synapses or specialized nerve endings form on a neuron. Enhancing synaptogenesis results in an increased number of synapses, while inhibiting synaptogenesis results in a decrease in the number of synapses, or a lack of increase where an increase would otherwise occur. By “augmentation,” “induction,” or “enhancement” of synaptogenesis as used herein, it is meant that the number of synapses formed is enhanced required in the specific situation.

As used herein, “agonists” may include proteins (i.e., polypeptides), nucleic acids, antibodies, or other molecules that affects a protein and/or molecule of interest, such as calcium channel subunit α₂δ. In some embodiments, an agonist may stimulate (e.g. increase) one or more activities or functions of a protein and/or molecule of interest.

As used herein, the term “thrombospondin” may refer to any one of the family of proteins which includes thrombospondins I, II, III, IV, and cartilage oligomeric matrix protein. Reference may also be made to one or more of the specific thrombospondins. Thrombospondin is a homotrimeric protein composed of three identical subunits (TSP1 and TSP2) or homopentameric protein composed of five identical subunits (TSPs 3-5) glycoprotein with disulfide-linked subunits of MW 180,000. It contains binding sites for thrombin, fibrinogen, heparin, fibronectin, plasminogen, plasminogen activator, collagen, laminin, calcium etc. and also contains domain homologues to procollagen, properdin, and epidermal growth factor (EGF). It functions in many cell adhesion and migration events, including platelet aggregation. Thrombospondin I (THBS1; also known as TSP1) has the Genbank accession number X04665 for the human DNA sequence and TSP1 human (P07996) for the human protein (see worldwide web at expasy.org/uniprot/P07996). It is a multimodular secreted protein that associates with the extracellular matrix and possesses a variety of biologic functions, including a potent angiogenic activity. Other thrombospondin genes include thrombospondins II (THBS2; 188061), III (THBS3; 188062), and IV (THBS4; 600715) with the corresponding protein sequences TSP1 Human (P07996), TSP2 Human (P35442); TSP3 Human (P49746), and TSP4 Human (P35443). Human thrombospondin 2 (THBS2; also known as TSP2) has the Genbank accession number L12350 (see worldwide web at expasy.org/uniprot/P35442) for the human sequence. It is very similar in sequence to THBS1. Human thrombospondin 3 (THBS3; also known as TSP3) has the Genbank accession number L38969 for the human sequence (see worldwide web at expasy.org/uniprot/P49476). The protein is clearly homologous to THBS1 and THBS2 in its COOH-terminal domains but substantially different in its NH2-terminal region, suggesting functional properties for THBS3 that are unique, but also related to those of THBS1 and THBS2. The 956-amino acid predicted protein is highly acidic, especially in the third quarter of the sequence which corresponds to 7 type III calcium binding repeats. Four type II EGF-like repeats are also present. The human THBS4 gene (also known as TSP4), Genbank accession number Z19585 for the human sequence (see worldwide web at expasy.org/uniprot/P35443), contains an RGD (arg-gly-asp) cell-binding sequence in the third type 3 repeat. It is a pentameric protein that binds to heparin and calcium. Cartilage oligomeric matrix protein (also known as TSP5), Genbank accession L32137 (see worldwide web at expasy.org/uniprot/P49474), is a 524-kD protein that is expressed at high levels in the territorial matrix of chondrocytes. The sequences indicate that it is a member of the thrombospondin gene family.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv), single chain (ScFv), mutants thereof, naturally occurring variants, fusion proteins comprising an antibody portion with an antigen recognition site of the required specificity, humanized antibodies, chimeric antibodies, and any other modified configuration of the immunoglobulin molecule that comprises an antigen recognition site of the required specificity. As used herein, the term also encompasses protein scaffolds for antibody mimics or scaffold-derived binding proteins that display properties such as small size, stability, and ease of production. These scaffold-based proteins comprise single domains of antibodies, immunoglobulin superfamily, protease inhibitors, helix-bundle proteins, disulphide-knotted peptides, protein A, the lipocalins, fibronectin domains, ankyrin consensus repeat domains, thioredoxins, and high disulfide density scaffold proteins. See e.g., U.S. Pat. No. 6,818,418, Lipovsek et al.; Skerra (2000) J Mol Recognit. 13(4):167-87; Skerra (2007) Current Opinion in Biotechnology, 18: 295-304; worldwide web at.amunix.com/Technology.html.

The term “monoclonal antibody” encompasses not only intact monoclonal antibodies and full-length monoclonal antibodies, but also fragments thereof (such as Fab, Fab′, F(ab′)₂, Fv), single chain (ScFv), mutants thereof, fusion proteins comprising an antibody portion, humanized monoclonal antibodies, chimeric monoclonal antibodies, diabodies, linear antibodies, and any other modified configuration of the immunoglobulin molecule that comprises an antigen recognition site of the required specificity and the ability to bind to an antigen. It is not intended to be limited as regards to the source of the antibody or the manner in which it is made (e.g., by hybridoma, phage selection, recombinant expression, transgenic animals, etc.).

“Humanized” antibodies refer to a molecule having an antigen-binding site that is substantially derived from an immunoglobulin from a non-human species and the remaining immunoglobulin structure of the molecule based upon the structure and/or sequence of a human immunoglobulin. The antigen-binding site may comprise either complete variable domains fused onto constant domains or only the complementarity determining regions (CDRs) grafted onto appropriate framework regions in the variable domains. Antigen binding sites may be wild type or modified by one or more amino acid substitutions, e.g., modified to resemble human immunoglobulin more closely. Some forms of humanized antibodies preserve all CDR sequences (for example, a humanized mouse antibody which contains all six CDRs from the mouse antibodies). Other forms of humanized antibodies have one or more CDRs (one, two, three, four, five, six) which are altered with respect to the original antibody, which are also termed one or more CDRs “derived from” one or more CDRs.

“Chimeric antibodies” refers to those antibodies wherein one portion of each of the amino acid sequences of heavy and light chains is homologous to corresponding sequences in antibodies derived from a particular species or belonging to a particular class, while the remaining segment of the chains is homologous to corresponding sequences in another. Typically, in these chimeric antibodies, the variable region of both light and heavy chains mimics the variable regions of antibodies derived from one species of mammals, while the constant portions are homologous to the sequences in antibodies derived from another. One clear advantage to such chimeric forms is that, for example, the variable regions can conveniently be derived from presently known sources using readily available hybridomas or B cells from non human host organisms in combination with constant regions derived from, for example, human cell preparations. While the variable region has the advantage of ease of preparation, and the specificity is not affected by its source, the constant region being human, is less likely to elicit an immune response from a human subject when the antibodies are injected than would the constant region from a non-human source. However, the definition is not limited to this particular example.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat, Sequences of Proteins of Immunological Interest, (5th ed., 1991, National Institutes of Health, Bethesda Md.)); and (2) an approach based on crystallographic studies of antigen-antibody complexes (Al-lazikani, (1997) J. Molec. Biol. 273:927-948)). As used herein, a CDR may refer to CDRs defined by either approach or by a combination of both approaches.

A “constant region” of an antibody refers to the constant region of the antibody light chain or the constant region of the antibody heavy chain, either alone or in combination.

An antibody or a polypeptide “specifically binds” or “preferentially binds” to a target if it binds with greater affinity, avidity, more readily, and/or with greater duration than it binds to other substances. For example, an antibody or a polypeptide that specifically or preferentially binds to an epitope within a VWF-A domain of a calcium channel α2δ subunit is an antibody or a polypeptide that binds this VWF-A epitope with greater affinity, avidity, more readily, and/or with greater duration than it binds to other calcium channel α2δ subunit epitopes or non-calcium channel α2δ subunit epitopes. It is also understood by reading this definition that, for example, an antibody or a polypeptide that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, the term “labeled”, with regard to the antibody, is intended to encompass direct labeling of the antibody by coupling (i.e., physically linking) a detectable substance, such as a radioactive agent or a fluorophore (e.g. fluorescein isothiocyanate (FITC) or phycoerythrin (PE)) to the antibody, as well as indirect labeling of the probe or antibody by reactivity with a detectable substance.

As used herein, “substantially pure” refers to material that is at least 50% pure (i.e., free from contaminants), more preferably at least 90% pure, more preferably at least 95% pure, more preferably at least 98% pure, more preferably at least 99% pure.

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon an antibody, the polypeptides can occur as single chains or associated chains.

An “isolated” molecule (such as a nucleic acid molecule or a protein including an antibody) is a molecule that is identified and separated from at least one contaminant molecule with which it is ordinarily associated in the environment in which it was produced. Preferably, the isolated molecule is free of association with all components associated with the production environment. The isolated molecule is in a form other than in the form or setting in which it is found in nature. Isolated molecules therefore are distinguished from the molecules existing naturally in cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

“Pharmaceutically acceptable” buffers and salts include those derived from both acid and base addition salts of the above indicated acids and bases. Specific buffers and/or salts include histidine, succinate and acetate.

The term “pharmaceutical formulation” refers to a preparation that is in such form as to permit the biological activity of the active ingredient to be effective, and that contains no additional components that are unacceptably toxic to a subject to which the formulation would be administered. Such formulations are sterile.

By “neurological” or “cognitive” function as used herein, it is meant that the increase of synapses in the brain enhances the patient's ability to think, function, etc. In conditions where there is axon loss and regrowth, there may be recovery of motor and sensory abilities.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. An individual is successfully “treated”, for example, if one or more symptoms associated with the disease or condition are mitigated or eliminated.

As used herein, the term “prevention” includes providing prophylaxis with respect to occurrence or recurrence of a disease in an individual. An individual may be predisposed to the disease but has not yet been diagnosed with the disease.

An “effective amount” refers to at least an amount effective, at dosages and for periods of time necessary, to achieve the desired therapeutic or prophylactic result. An effective amount can be provided in one or more administrations.

A “therapeutically effective amount” is at least the minimum concentration required to effect a measurable improvement of a particular disorder. A therapeutically effective amount herein may vary according to factors such as the disease state, age, sex, and weight of the patient, and the ability of the antibody to elicit a desired response in the individual. A therapeutically effective amount is also one in which any toxic or detrimental effects are outweighed by the therapeutically beneficial effects.

A “prophylactically effective amount” refers to an amount effective, at the dosages and for periods of time necessary, to achieve the desired prophylactic result. Typically but not necessarily, since a prophylactic dose is used in subjects prior to or at an earlier stage of disease, a prophylactically effective amount may be less than a therapeutically effective amount.

“Chronic” administration refers to administration of the medicament(s) in a continuous as opposed to acute mode, so as to maintain the initial therapeutic effect (activity) for an extended period of time.

“Intermittent” administration refers to treatment that is not consecutively done without interruption, but rather is cyclic in nature.

As used herein, administration “in conjunction” includes simultaneous administration and/or administration at different times. Administration in conjunction also encompasses administration as a co-formulation or administration as separate compositions, including at different dosing frequencies or intervals, and using the same route of administration or different routes of administration.

An “individual” or “subject” refers a mammal, including humans, domestic and farm animals, and zoo, sport, or pet animals, such as chimpanzees and other apes and monkey species, dogs, horses, rabbits, cattle, pigs, goats, sheep, hamsters, guinea pigs, gerbils, mice, ferrets, rats, cats, and the like. Preferably, the individual is human. The term does not denote a particular age or gender.

The term “about” as used herein refers to the usual error range for the respective value readily known to the skilled person in this technical field. Reference to “about” a value or parameter herein includes (and describes) embodiments that are directed to that value or parameter per se.

As used herein the singular forms “a”, “and”, and “the” include plural referents unless the context clearly dictates otherwise.

It is understood that aspects and embodiments of the invention described herein include “consisting” and/or “consisting essentially of” aspects and embodiments.

Compositions and Methods of Inducing Synaptogenesis and/or Axonal Growth

The present invention provides isolated antibodies and polypeptides (which may or may not be an antibody) that specifically bind to an epitope in a von Willebrand Factor A (VWF-A) domain of a calcium channel subunit selected from the group consisting of α2δ1, α2δ2, α2δ3, and α2δ4 and induces synaptogenesis of a neuronal cell expressing the calcium channel subunit.

The present invention also provides methods for inducing synaptogenesis and neurite outgrowth (axonal and/or dendritic growth) in an individual. The methods comprise administering an effective dose of an antibody or a polypeptide that binds to an epitope in a VWF-A domain of a calcium channel subunit α2δ (e.g., α2δ1, α2δ2, α2δ3, and α2δ4) induces synaptogenesis of a neuronal cell expressing the calcium channel subunit. Axonal and neuronal survival and functionality are dependent on the presence of active synapses and inducing synaptogenesis is anticipated to promote the functionality of axon and dendrites and the survival of neurons.

Calcium Channel, Alpha-2/Delta Subunit

Calcium channels mediate the influx of calcium ions into the cell upon membrane polarization and consist of a complex of alpha-1 (α1), alpha-2/delta (α2δ), beta (β), and gamma (γ) subunits in a 1:1:1:1 ratio. The al subunit determines the main biophysical properties of the channel and is modulated by the other subunits. The intracellular β subunit is responsible for trafficking. The α₂δ is a transmembrane subunit.

The alpha-2/delta subunit of skeletal muscle and brain voltage-dependent calcium channels are encoded by a family of genes designated CACNA2D. Alternative names for these molecules include alpha2delta subunits; Cacna2d; calcium channel alpha2/delta; calcium channel, voltage-dependent, alpha2/delta subunits; calcium channel, voltage-dependent, alpha2/delta subunits; Cch12a; and voltage-dependent calcium channel alpha2/delta. CACNA2D proteins alter the properties of pore-forming alpha-1 subunits of voltage-gated calcium channels, and are posttranslationally processed into 2 peptides, an alpha-2 subunit and a delta subunit, that are held together by a disulfide bond. The alpha-2/delta proteins are encoded by at least 4 different genes: CACNA2D1 (α₂δ1), CACNA2D2 (α₂δ2), CACNA2D3 (α₂δ3), and CACNA2D4 (α₂δ4) (see, for example Schleithoff., 1999 Genomics 61: 201-209; and Field, (2006) Proc. Nat. Acad. Sci. 103: 17537-17542, herein specifically incorporated by reference). The genetic sequences and protein sequences are publicly available. For example, genetic sequence for α₂δ1 is at Genbank, accession number BC117470; and protein sequence for α₂δ1 is at Genbank accession number P54289 (SEQ ID NO:1); protein sequence for α₂δ2 is at Genbank accession number KIAA0558 (SEQ ID NO.2); protein sequence for α₂δ3 is at Genbank accession number Q8IZS8 (SEQ ID NO:3); and protein sequence for α₂δ4 is at Genbank accession number Q7Z3S7 (SEQ ID NO:4).

Iles, (1994) Hum. Molec. Genet. 3: 969-975 cloned and partially sequenced the CACNL2A gene. The CACNL2A is expressed in many neuronal and non neuronal tissues, including skeletal muscle, brain, heart, and lung (Cole, 2005). For example alpha-2/delta2 is expressed primarily in GABAergic neurons (Cole, 2005). A comparison of sequences of cDNAs representing the skeletal muscle and brain isoforms showed that they are encoded by a single gene. The “delta” portion, encoded by exons 37 to 40, is posttranscriptionally cleaved from the C-terminal “alpha” portion of the protein. The membrane-spanning region of the delta portion is encoded by exon 40. The CACNA2D1 gene undergoes alternative splicing at exons 19 and 24, corresponding to muscle and brain isoforms, respectively.

The topology of the α₂δ protein appears to generalize for all four α₂δ family members. They are all predicted to be type 1 transmembrane proteins, because all have a hydrophobic region in the C-terminus (CT) that is likely to be a transmembrane domain. Each member of the α2δ protein family is translated from a single gene product, which gets post-translationally cleaved into α2 and δ parts that remain associated via disulfide bridges. The α2 portion of the protein is entirely extracellular while the δ portion has a small extracellular part that is attached to α2, and a transmembrane domain with a very short cytoplasmic tail that tethers the whole molecule to the membrane (Davies, Trends in Pharmacol. Sci. 28:220-228, 2007).

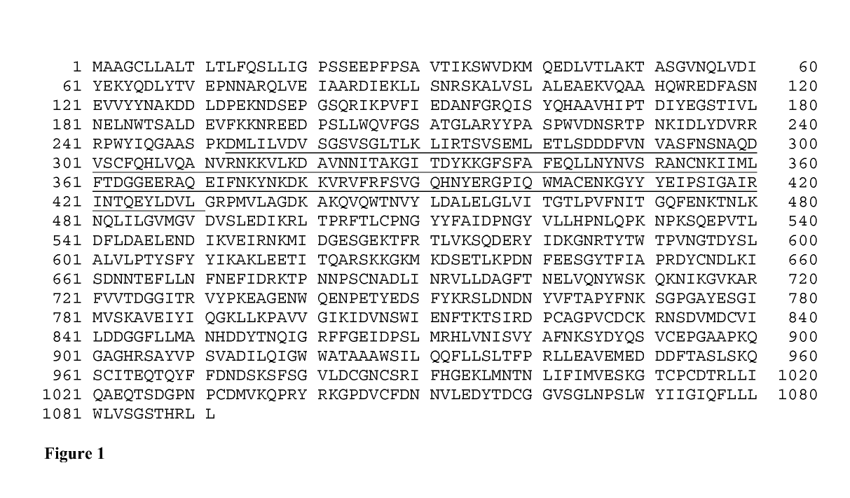

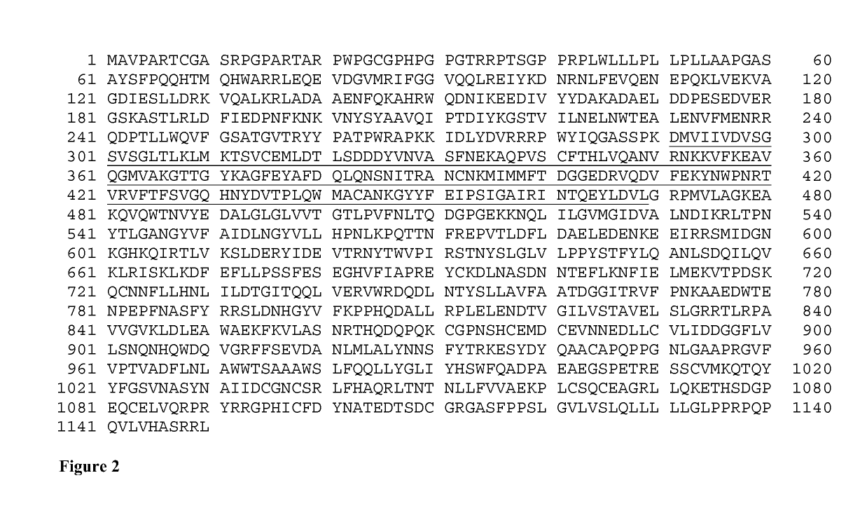

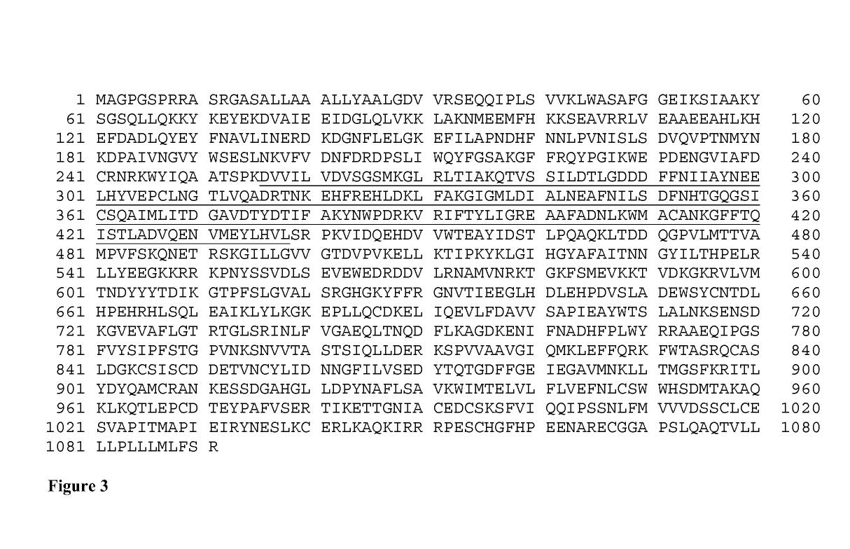

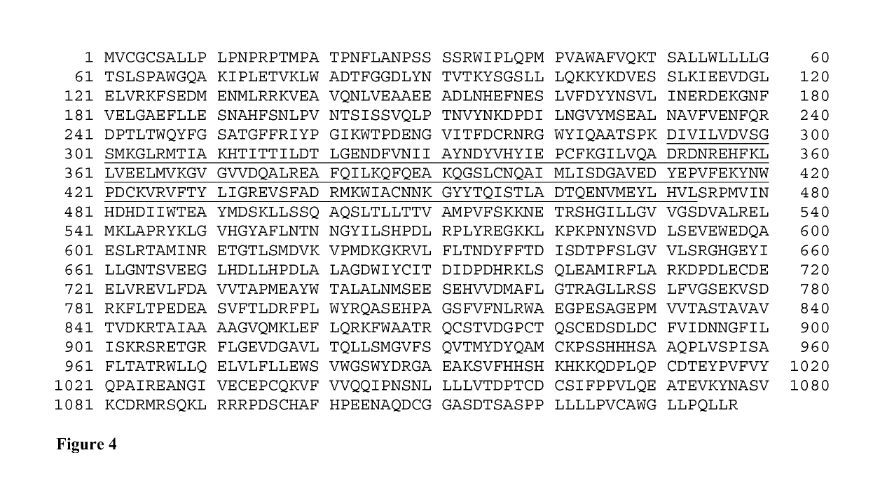

von Willebrand factor A (VWF-A) domain in calcium channel α₂δ subunits (such as human calcium channel α₂δ subunits) are known. For example, amino acids from about 253 to about 430 of α₂δ1 (as underlined in FIG. 1 of SEQ ID NO:1), amino acids from about 291 to about 469 of α₂δ2 (as underlined in FIG. 2 of SEQ ID NO:2), amino acids from about 256 to about 438 of α₂δ3 (as underlined in FIG. 3 of SEQ ID NO:3), and amino acids from about 291 to about 473 of α₂δ4 (as underlined in FIG. 4 of SEQ ID NO:4) have been identified as the VWF-A domain in these molecules. The amino acid positions are based on the unprocessed precursor protein with signal sequence.

Antibodies and Polypeptides Against VWF-A Domains of Calcium Channel α2δ Subunits

The present invention provides antibodies and polypeptides that specifically bind to an epitope in a VWF-A domain of a calcium channel subunit α₂δ. In some embodiments, the α₂δ subunit is α₂δ₁, α₂δ₂, α₂δ₂, or α₂δ₄. In some embodiments, the antibody or polypeptide binds to the calcium channel α₂δ subunit and induces synaptogenesis of neuronal cells expressing the calcium channel α₂δ subunit. In some embodiments, the antibody or polypeptide may bind to and activate more than one type of α₂δ subunits because of the homology between the molecules. For example, the antibody or polypeptide described herein may bind to and activate both α₂δ₁ and α₂δ₂. In some embodiments, the antibody or polypeptide may bind to and activate both α₂δ₁ and α₂δ₃. In some embodiments, the antibody or polypeptide may bind to and activate both α₂δ₁ and α₂δ₄. Binding affinity of the antibody or polypeptide to different type of subunit may be similar or different. The antibody or polypeptide may have higher affinity to one type of subunit as compared to a different type of subunit.

The binding affinity (such as K_{D}) of the antibody or polypeptide to a calcium channel subunit α₂δ may be less than any of about 500 nM, about 400 nM, about 300 nM, about 200 nM, about 100 nM, about 50 nM, about 10 nM, about 1 nM, about 500 pM, about 100 pM, or about 50 pM. As is well known in the art, binding affinity can be expressed as K_{D}, or dissociation constant, and an increased binding affinity corresponds to a decreased K_{D}. One way of determining binding affinity of an antibody or a polypeptide to its target is by measuring binding affinity of monofunctional fragment, such as Fab fragments of the antibody. The affinity of an Fab fragment of an antibody can be determined by surface plasmon resonance (BIAcore3000™ surface plasmon resonance (SPR) system, BIAcore, INC, Piscaway N.J.) and ELISA. Kinetic association rates (kₒₙ) and dissociation rates (k_{off}) (generally measured at 25° C.) are obtained; and equilibrium dissociation constant (K_{D}) values are calculated as k_{off}/kₒₙ.

In some embodiments, the antibody binds to an epitope within amino acids of about 253 to about 430 of human α₂δ₁ (as underlined in FIG. 1 of SEQ ID NO:1). In some embodiments, the antibody binds to an epitope within amino acids of about 291 to about 469 of human α₂δ₂ (as underlined in FIG. 2 of SEQ ID NO:2). In some embodiments, the antibody binds to an epitope within amino acids of about 253 to about 438 of human α₂δ₃ (as underlined in FIG. 3 of SEQ ID NO:3). In some embodiments, the antibody binds to an epitope within amino acids of about 291 to about 473 of human α₂δ₄ (as underlined in FIG. 4 of SEQ ID NO:4) In some embodiments, the antibody binds to a peptide having the amino acid sequence of SEQ ID NO:6 (MZp110). In some embodiments, the antibody binds to a peptide having the amino acid sequence of SEQ ID NO:5 (MZp 109).

The antibodies described herein can encompass polyclonal antibodies, monoclonal antibodies, antibody fragments (e.g., Fab, Fab′, F(ab′)₂, Fv, Fc, etc.), chimeric antibodies, single chain (ScFv), bispecific antibodies, mutants thereof, fusion proteins comprising an antibody portion, and any other modified configuration of the immunoglobulin molecule that comprises an antigen recognition site of the required specificity. The antibodies may be murine, rat, camel, human, or any other origin (including humanized antibodies). In some embodiments, the antibody is a polyclonal antibody. In some embodiments, the antibody is a monoclonal antibody.

Methods of generating antibodies to an epitope within a VWF-A domain of a calcium channel subunit α₂δ is known in the art. For example, as described in Example 1, peptides within a VWF-A domain may be used for immunization, and antibodies that specifically bind to the peptide and the calcium channel subunit α₂δ may be identified by ELISA, Western blot and fluorescent immunostaining as described in Examples 1 and 2. Methods for treating the synaptic inducing activities of antibodies or polypeptides are also known in the art and described in Example 2 herein.

Antibodies to an epitope within a VWF-A domain of a calcium channel subunit α₂δ may also be generated by screening an antibody library, such as a phage display library. The antibodies in the library may be human antibodies, humanized antibodies, chimeric antibodies. The antibodies in the library may also be single chain antibodies or single domain antibodies.

The invention also provides antibody 5A5, and antibodies and polypeptides derived from antibody 5A5. In accordance with the Budapest Treaty, a hybridoma which produces antibody 5A5 has been deposited in the American Type Culture Collection (ATCC) 10801 University Blvd., Manassas Va. 20110-2209 on ______ with a Patent Deposit Designation of ______. The invention provides an antibody or a polypeptide comprising a fragment or a region of the antibody 5A5. In one embodiment, the fragment is a light chain of antibody 5A5. In another embodiment, the fragment is a heavy chain of the antibody 5A5. In yet another embodiment, the fragment contains one or more variable regions from a light chain and/or a heavy chain of antibody 5A5. In yet another embodiment, the fragment contains one, two, or three CDRs from the light chain and/or one, two, three CDRs from the heavy chain of antibody 5A5. In some embodiments, the antibody is a humanized version of antibody 5A5. In some embodiments, the antibody comprises one or more CDRs derived from antibody 5A5 that are greater than or about 85%, greater than or about 86%, greater than or about 87%, greater than or about 88%, greater than or about 89%, greater than or about 90%, greater than or about 91%, greater than or about 92%, greater than or about 93%, greater than or about 94%, greater than or about 95%, greater than or about 96%, greater than or about 97%, greater than or about 98%, or greater than or about 99% identical to one or more, two or more, three or more, four or more, five or more, or six CDRs of antibody 5A5.

The invention also provides antibody 3B4, and antibodies and polypeptides derived from antibody 3B4. In accordance with the Budapest Treaty, a hybridoma which produces antibody 3B4 has been deposited in the American Type Culture Collection (ATCC) 10801 University Blvd., Manassas Va. 20110-2209 on ______ with a Patent Deposit Designation of ______. The invention provides an antibody or a polypeptide comprising a fragment or a region of the antibody 3B4. In one embodiment, the fragment is a light chain of antibody 3B4. In another embodiment, the fragment is a heavy chain of the antibody 3B4. In yet another embodiment, the fragment contains one or more variable regions from a light chain and/or a heavy chain of antibody 3B4. In yet another embodiment, the fragment contains one, two, or three CDRs from the light chain and/or one, two, three CDRs from the heavy chain of antibody 3B4. In some embodiments, the antibody is a humanized version of antibody 3B4. In some embodiments, the antibody comprises one or more CDRs derived from antibody 3B4 that are greater than or about 85%, greater than or about 86%, greater than or about 87%, greater than or about 88%, greater than or about 89%, greater than or about 90%, greater than or about 91%, greater than or about 92%, greater than or about 93%, greater than or about 94%, greater than or about 95%, greater than or about 96%, greater than or about 97%, greater than or about 98%, or greater than or about 99% identical to one or more, two or more, three or more, four or more, five or more, or six CDRs of antibody 3B4.

In some embodiments, the CDR is a Kabat CDR. In other embodiments, the CDR is a Chothia CDR. In other embodiments, the CDR is a combination of a Kabat and a Chothia CDR (also termed “combined CDR” or “extended CDR”). In other words, for any given embodiment containing more than one CDR, the CDRs may be any of Kabat, Chothia, and/or combined.

The invention also provides polypeptides that specifically bind an epitope in a VWF-A domain of a calcium channel subunit α₂δ. In some embodiments, the polypeptide is derived from any of the antibodies described herein. In some embodiments, the polypeptide is a protein scaffold for antibody mimics or scaffold-derived binding proteins that display properties like small size, stability, and ease of production. These include single domains of antibodies or the immunoglobulin superfamily, protease inhibitors, helix-bundle proteins, disulphide-knotted peptides, protein A, the lipocalins, fibronectin domains, ankyrin consensus repeat domains, thioredoxins, and high disulfide density scaffold proteins. See e.g., U.S. Pat. No. 6,818,418, Lipovsek et al.; Skerra, J. Mol. Recognit. 13(4):167-87, 2000; Skerra, Current Opinion in Biotechnology 18:295-304, 2007; world wide web at amunix.com/Technology.html.

The invention also provides methods for screening a candidate antibody or polypeptide for activity in enhancing synaptogenesis. In some embodiments, the method comprises a) contacting neuronal cells expressing a calcium channel subunit selected from the group consisting of α2δ1, α2δ2, α2δ3, and α2δ4 with an candidate antibody or polypeptide that specifically binds to an epitope in a von Willebrand Factor A (VWF-A) domain of the calcium channel subunit; and b) quantitating formation of synapses in the cell culture in the presence of the candidate antibody or polypeptide. In some embodiments, the method comprises: a) measuring binding of a candidate antibody or polypeptide to an α2δ polypeptide (e.g., α2δ1 polypeptide); and b) quantitating formation of synapses in a neural cell culture in the presence of the candidate antibody or polypeptide if the candidate antibody binds to the α2δ polypeptide (e.g., α2δ1 polypeptide), wherein an increased formation of synapses in the presence the candidate antibody or polypeptide as compared to the formation of synapses in the absence of the candidate antibody or polypeptide indicates that the candidate antibody or polypeptide has the activity in enhancing synaptogenesis.

Candidate antibodies or polypeptides are screened for the ability to induce synaptogenesis. Antibodies or polypeptides may be screened against an epitope in a VWF-A domain of a calcium channel subunit α2δ. Antibody or polypeptide screening may be performed using an in vitro model, a cell expressing the polypeptide, including a genetically altered cell or animal, or purified protein. A wide variety of assays may be used for this purpose. In one embodiment, antibodies or polypeptides are initially tested for binding to calcium channel subunit α2δ (e.g., calcium channel subunit α2δ1). The compounds may then be further tested for functional activity in a biological model, e.g. an in vitro culture system, as described below, an animal model, etc. In some embodiments, the number of synapse increase is at least about any of 10%, 20%, 30%, 40%, 50%, 75%, 100%, 150%, or 200% on cells treated with the antibodies or polypeptides relative to a control in an in vitro assay or in an in vivo assay.

For example, candidate antibodies or polypeptides may be identified by known pharmacology, by structure analysis, by rational drug design using computer based modeling, by binding assays, and the like. Various in vitro models may be used to determine whether antibodies or polypeptides bind to an epitope in a VWF-A domain. Such candidate antibodies or polypeptides are used to contact neurons in an environment permissive for synaptogenesis. Neuronal cells that may be used include motor neurons, dopaminergic neurons, retinal ganglia neurons, cholinergic neurons, GABAnergic (γ-aminobutyric acid producing) neurons, sertonergic neurons, sympathetic and para sympathetic neurons, sensory neurons, interneurons, neurons that use glutamate, aspartate, serine, glycine, dopamine (DA), norepinephrine (noradrenaline; NE, NA), epinephrine (adrenaline), histamine, serotonin (SE, 5-HT), melatonin, acetylcholine (ACh), adenosine, anandamide, nitric oxide or any of the other known neurotransmitters (e.g., neurotransmitters-Dorland's Medical Dictionary), or any other neurons expressing a calcium channel subunit α2δ. These neurons may be derived from a mouse, a rat, or a human, and may be further purified. Such antibodies or polypeptides may be further tested in an in vivo model for enhanced synaptogenesis.

Synaptogenesis is quantitated by administering the candidate antibodies or polypeptides to neurons in culture, and determining the presence of synapses in the absence or presence of the antibodies or polypeptides. In one embodiment of the invention, the neurons are a primary culture, e.g. of retinal ganglion neurons (RGCs). Purified populations of RGCs are obtained by conventional methods, such as sequential immunopanning. The cells are cultured in suitable medium, which will usually comprise appropriate growth factors, e.g. CNTF; BDNF; etc. As a positive control, soluble thrombospondin, e.g. TSP1, TSP2, etc. may be added to certain wells. The neural cells, e.g. RCGs, are cultured for a period of time sufficient allow robust process outgrowth and then cultured with a candidate antibody or polypeptide for a period of about 1 day to 1 week, to allow synapse formation. For synapse quantification, cultures are fixed, blocked and washed, then stained with antibodies specific synaptic proteins, e.g. synaptotagmin, etc. and visualized with an appropriate reagent, as known in the art. Analysis of the staining may be performed microscopically. In one embodiment, digital images of the fluorescence emission are with a camera and image capture software, adjusted to remove unused portions of the pixel value range and the used pixel values adjusted to utilize the entire pixel value range. Corresponding channel images may be merged to create a color (RGB) image containing the two single-channel images as individual color channels. Co-localized puncta can be identified using a rolling ball background subtraction algorithm to remove low-frequency background from each image channel. Mean number, area, minimum and maximum pixel intensities, and mean pixel intensities for all synaptotagmin, PSD-95, and colocalized puncta in the image are recorded and saved to disk for analysis.

In some embodiments, a plurality of assay mixtures are run in parallel with different antibodies or polypeptides concentrations to obtain a differential response to the various concentrations. Typically one of these concentrations serves as a negative control, i.e. at zero concentration or below the level of detection.

Candidate antibodies or polypeptides are obtained from a wide variety of sources including libraries of synthetic or natural compounds. For example, numerous means are available for random and directed synthesis of antibodies and polypeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example.

Candidate antibodies or polypeptides that are initially identified by any screening methods can be further tested to validate the apparent activity. The basic format of such methods involves administering a lead antibody or polypeptide identified during an initial screen to an animal that serves as a model for humans and then determining the effects on synaptogenesis. The animal models utilized in validation studies generally are mammals. Specific examples of suitable animals include, but are not limited to, primates, mice, and rats. Examples of animal models for traumatic brain injury, stroke, amyotrophic lateral sclerosis (ALS), and Alzheimer's disease are described in Example 3.

Polynucleotides, Vectors and Host Cells

The invention also provides isolated polynucleotides encoding the antibodies and polypeptides of the invention, and vectors and host cells comprising the polynucleotide.

The invention provides polynucleotides (or compositions, including pharmaceutical compositions, comprising the polynucleotides), comprising a nucleotide sequence encoding any of the following: (a) antibody 5A5; (b) a fragment or a region of antibody 5A5; (c) a light chain of the antibody 5A5; (d) a heavy chain of antibody 5A5; (e) one or more variable region(s) from a light chain and/or a heavy chain of the antibody 5A5; (f) one or more CDR(s) (one, two, three, four, five or six CDRs) of antibody 5A5; (g) the three CDRs from the light chain of antibody 5A5; (h) the three CDRs from the heavy chain of antibody 5A5; (i) the three CDRs from the light chain and three CDRs from the heavy chain of antibody 5A5; or (j) an antibody comprising any of (b) to (i).

The invention provides polynucleotides (or compositions, including pharmaceutical compositions comprising the polynucleotides), comprising a nucleotide sequence encoding any of the following: (a) antibody 3B4; (b) a fragment or a region of antibody 3B4; (c) a light chain of the antibody 3B4; (d) a heavy chain of antibody 3B4; (e) one or more variable region(s) from a light chain and/or a heavy chain of the antibody 3B4; (f) one or more CDR(s) (one, two, three, four, five or six CDRs) of antibody 3B4; (g) the three CDRs from the light chain of antibody 3B4; (h) the three CDRs from the heavy chain of antibody 3B4; (i) the three CDRs from the light chain and three CDRs from the heavy chain of antibody 3B4; or (j) an antibody comprising any of (b) to (i).

In another aspect, the invention provides vectors comprising any of the polynucleotides of the invention. In some embodiments, the vector comprises an expression vector comprising a polynucleotide encoding any antibodies or polypeptides as described herein. Expression vectors, and administration of polynucleotide compositions are further described herein. In another aspect, the invention provides a method of making any of the polynucleotides described herein.

Polynucleotides complementary to any such sequences are also encompassed by the present invention. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al. (1989).

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston (1994).

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., (1989), for example.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp 18, mp 19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The invention also provides host cells comprising any of the polynucleotides described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis). Preferably, the host cells express the cDNAs at a level of about 5 fold higher, more preferably 10 fold higher, even more preferably 20 fold higher than that of the corresponding endogenous antibody or protein of interest, if present, in the host cells. A cell overexpressing the antibody or protein of interest can be identified.

The invention also provides an isolated cell line that produces antibody 5A5 or a progeny thereof, which cell line has ATCC Patent Deposit Designation No. ______. The invention also provides an isolated cell line that produces antibody 3B4 or a progeny thereof, which cell line has ATCC Patent Deposit Designation No. ______.

The invention also provides methods of producing an antibody or polypeptide described herein, the method comprising culturing a host cell that produces the antibody or the poplypeptide, and recovering the antibody or polypeptide from the cell culture. In some embodiments, one or more polynucleotides encoding the antibody or polypeptide have been introduced into the host cells.

Inducing Synaptogenesis

Synaptogenesis is a dynamic process. During development, more synapses are established than ultimately will be retained. Therefore, the elimination of excess synaptic inputs is a critical step in synaptic circuit maturation. Synapse elimination is a competitive process that involves interactions between pre- and postsynaptic partners. In the central nervous system (CNS), as with the neuromuscular junction (NMJ), a developmental, activity-dependent remodeling of synaptic circuits takes place by a process that may involve the selective stabilization of coactive inputs and the elimination of inputs with uncorrelated activity. The anatomical refinement of synaptic circuits occurs at the level of individual axons and dendrites by a dynamic process that involves rapid elimination of synapses. As axons branch and remodel, synapses form and dismantle with synapse elimination occurring rapidly.

Synapses are asymmetric communication junctions formed between two neurons, or, at the NMJ between a neuron and a muscle cell. Chemical synapses enable cell-to-cell communication via secretion of neurotransmitters, whereas in electrical synapses signals are transmitted through gap junctions, specialized intercellular channels that permit ionic current flow. In addition to ions, other molecules that modulate synaptic function (such as ATP and second messenger molecules) can diffuse through gap junctional pores. At the mature NMJ, pre- and postsynaptic membranes are separated by a synaptic cleft containing extracellular proteins that form the basal lamina. Synaptic vesicles are clustered at the presynaptic release site, transmitter receptors are clustered in junctional folds at the postsynaptic membrane, and glial processes surround the nerve terminal.

In some embodiments of any of the methods described herein, synapse formation may be increased. In some embodiments, synapses are increased due to increased new synapse formation. In some embodiments, synapses are increased due to increased synapse maintenance. In some embodiments, the synapses are at the neuromuscular junction. In some embodiments, the synapses comprise or consist of excitatory synapses. In some embodiments, the synapses are VGlut2 positive excitatory synapses. In some embodiments, the synapses are VGlut1 positive excitatory synapses. In some embodiments, the synapse formation is increased after synapse loss due to senescence. In some embodiments, the synapse formation is increase after synapses loss due to injury. In some embodiments, sense organ function is restored after loss due to injury, senescence or a genetic disorder.

Diseases and Conditions of Interest

The methods described herein may be used to induce synaptogenesis in a variety of diseases and conditions in which increase in the number of synapses would be beneficial. The conditions of interest that may be benefited (e.g., treated and/or prevented) from the present methods include, but not limited to, senescence, stroke, spinal cord injury, Alzheimer's disease, Parkinson/s disease, multiple sclerosis, aggression, amyotrophic lateral sclerosis, autism, neuropathy, mascular dystrophy, Huntington disease, alcoholism, Alexander's disease, Alper's disease ataxia telangiectasia, Batten disease (also known as Spielmeyer-Vogt-Sjogren-Batten disease), bovine spongiform encephalopathy (BSE), canavan disease, Cockayne syndrome, corticobasal degeneration, chemotherapy induced neuropathy, Creutzfeldt-Jakob disease, diabetic neuropathy, denervation atrophy, depression, Down syndrome, hearing loss, glaucoma, HIV-associated dementia, Kennedy's disease, Krabbe's disease, Lewy body dementia, Machado-Joseph disease (Spinocerebellar ataxia type 3), macular degeneration, multiple system atrophy, myofiber atrophy, narcolepsy, neuroborreliosis, Pelizaeus-Merzbacher Disease, primary lateral sclerosis, poliomyelitis virus, Prion disease, Progressive Supranuclear Palsy, Refsum's disease, Sandhoff's disease, Schilder's disease, subacute combined degeneration of spinal cord secondary to pernicious anaemia, schizophrenia, Spielmeyer-Vogt-Sjogren-Batten disease (also known as Batten disease), spinocerebellar ataxia (multiple types with varying characteristics), spinal muscular atrophy, Steele-Richardson-Olszewski disease, Tabes dorsalis, West Niles virus, as well as promoting new synaptogenesis in repair and regeneration of injured CNS after stroke or spinal cord injury. Such conditions benefit from administration of the antibody or the polypeptide as described in the present invention, which increase, or enhance, the development of synapses or sense organs. In some instances, where there has been neuronal loss, it may be desirable to enhance neurogenesis as well, e.g. through administration of antibodies or regimens that increase neurogenesis, transplantation of neuronal progenitors, etc. In some embodiments, one or more symptoms associated with the disease or condition are mitigated or eliminated.

Patients can suffer neurological and functional deficits after stroke, CNS injury, and neurodegenerative disease. The findings of the present invention provide a means to modulate synapse formation and to improve function after CNS damage or degeneration. The induction of neural connections induced by promoting synaptogenesis will promote functional improvement and will increase neuronal survival after stroke, injury, aging and neurodegenerative disease. The amount of increased synaptogenesis may comprise at least a measurable increase relative to a control lacking such treatment, for example at least a 10% increase, at least a 20% increase, at least a 50% increase, or more. In some embodiments, the number of synapses may be increased at least about any of 10%, 20%, 30%, 40%, 50%, or 60%. In some embodiments, the synapses are at the neuromuscular junction. In some embodiments, the synapses comprise or consist of excitatory synapses. In some embodiments, the synapses are VGlut2 positive excitatory synapses. In some embodiments, the synapses are VGlut1 positive excitatory synapses. In some embodiments, synapses are increased due to increased new synapse formation. In some embodiments, synapses are increased due to increased synapse maintenance.

In some embodiments of any of the methods described herein, an individual or subject may have suffered synapse loss as a result of senescence. In some embodiments, the individual or subject may have suffered synapse loss as a result of Alzheimer's disease, Parkinson's disease, ALS, multiple sclerosis, Huntington disease, Down syndrome, spinal mascular atrophy, spinal cord injury, Myofiber atrophy, denervation atrophy, or glaucoma. In some embodiments, an individual or subject may have suffered macular degeneration, a hearing loss, diabetic neuropathy, or chemotherapy induced neuropathy. In some embodiments, the individual or subject may have suffered synapse loss as a result of a psychiatric disorder selected from the group consisting of acute stress disorder, aggression, agoraphobia, autism, dissociative amnesia, anorexia nervosa, bipolar disorder, body dysmorphic disorder, brief psychotic disorder, bulimia nervosa, conversion disorder, cyclothymic disorder, delusional disorder, depersonalization disorder, depression, dissociative identity disorder (DID), dysparenunia, dysthymic disorder, male erectile disorder, generalized anxiety disorder, impotence, pain disorder, panic disorder, phobias, posttraumatic stress disorder, schizoaffective disorder, schizophrenia, schizophreniform, shared psychotic disorder, and substance abuse. In some embodiments, the individual may have suffered synapse loss due to injury such as spinal cord injury or central nervous system injury. In some embodiments, the individual or subject may have suffered synapse loss as a result of a viral infection selected from the group consisting of poliomyelitis virus, West Nile Virus, and Human immunodeficiency virus (HIV). In some embodiments, the individual or subject may have suffered synapse loss as a result of a Prion disease selected from the group consisting of kuru, Creutzfeldt-Jakob disease, variant Creutzfeldt-Jakob disease, Gerstmann-Sträussler-Scheinker syndrome, and fatal familial insomnia.

The term “stroke” broadly refers to the development of neurological deficits associated with impaired blood flow to the brain regardless of cause. Potential causes include, but are not limited to, thrombosis, hemorrhage and embolism. Current methods for diagnosing stroke include symptom evaluation, medical history, chest X-ray, ECG (electrical heart activity), EEG (brain nerve cell activity), CAT scan to assess brain damage and MRI to obtain internal body visuals. Thrombus, embolus, and systemic hypotension are among the most common causes of cerebral ischemic episodes. Other injuries may be caused by hypertension, hypertensive cerebral vascular disease, rupture of an aneurysm, an angioma, blood dyscrasias, cardiac failure, cardiac arrest, cardiogenic shock, septic shock, head trauma, spinal cord trauma, seizure, bleeding from a tumor, or other blood loss.

By “ischemic episode” is meant any circumstance that results in a deficient supply of blood to a tissue. When the ischemia is associated with a stroke, it can be either global or focal ischemia, as defined below. The term “ischemic stroke” refers more specifically to a type of stroke that is of limited extent and caused due to blockage of blood flow. Cerebral ischemic episodes result from a deficiency in the blood supply to the brain. The spinal cord, which is also a part of the central nervous system, is equally susceptible to ischemia resulting from diminished blood flow.

Senescence refers to the effects or the characteristics of increasing age, particularly with respect to the diminished ability of somatic tissues to regenerate in response to damage, disease, and normal use. Alternatively, aging may be defined in terms of general physiological characteristics. The rate of aging is very species specific, where a human may be aged at about 50 years; and a rodent at about 2 years. In general terms, a natural progressive decline in body systems starts in early adulthood, but it becomes most evident several decades later. One arbitrary way to define old age more precisely in humans is to say that it begins at conventional retirement age, around about 60, around about 65 years of age. Another definition sets parameters for aging coincident with the loss of reproductive ability, which is around about age 45, more usually around about 50 in humans, but will, however, vary with the individual. Loss of synaptic function may be found in aged individuals, such as mild cognitive deficient.

Among the aged, Alzheimer's disease is a serious condition. Alzheimer's disease is a progressive, inexorable loss of cognitive function associated with an excessive number of senile plaques in the cerebral cortex and subcortical gray matter, which also contains β-amyloid and neurofibrillary tangles consisting of tau protein. The common form affects persons >60 yr old, and its incidence increases as age advances. It accounts for more than 65% of the dementias in the elderly.

The cause of Alzheimer's disease is not known. The disease runs in families in about 15 to 20% of cases. The remaining, so-called sporadic cases have some genetic determinants. The disease has an autosomal dominant genetic pattern in most early-onset and some late-onset cases but a variable late-life penetrance. Environmental factors are the focus of active investigation.

In the course of the disease, neurons are lost within the cerebral cortex, hippocampus, and subcortical structures (including selective cell loss in the nucleus basalis of Meynert), locus caeruleus, and nucleus raphae dorsalis. Cerebral glucose use and perfusion is reduced in some areas of the brain (parietal lobe and temporal cortices in early-stage disease, prefrontal cortex in late-stage disease). Neuritic or senile plaques (composed of neurites, astrocytes, and glial cells around an amyloid core) and neurofibrillary tangles (composed of paired helical filaments) play a role in the pathogenesis of Alzheimer's disease. Senile plaques and neurofibrillary tangles occur with normal aging, but they are much more prevalent in persons with Alzheimer's disease.

The essential features of dementia are impairment of short-term memory and long-term memory, abstract thinking, and judgment; other disturbances of higher cortical function; and personality change. Progression of cognitive impairment confirms the diagnosis, and patients with Alzheimer's disease do not improve.

The methods of the invention also find use in combination with cell or tissue transplantation to the central nervous system, where such grafts include neural progenitors such as those found in fetal tissues, neural stem cells, embryonic stem cells or other cells and tissues contemplated for neural repair or augmentation. Neural stem/progenitor cells have been described in the art, and their use in a variety of therapeutic protocols has been widely discussed. For example, inter alia, U.S. Pat. Nos. 6,638,501, Bjornson et al.; U.S. Pat. No. 6,541,255, Snyder et al.; U.S. Pat. No. 6,498,018, Carpenter; U.S. Patent Application 20020012903, Goldman et al.; Palmer et al. (2001) Nature 411(6833):42-3; Palmer et al. (1997) Mol Cell Neurosci. 8(6):389-404; Svendsen et al. (1997) Exp. Neurol. 148(1):135-46 and Shihabuddin (1999) Mol Med Today 5(11):474-80; each herein specifically incorporated by reference.

Neural stem and progenitor cells can participate in aspects of normal development, including migration along well-established migratory pathways to disseminated CNS regions, differentiation into multiple developmentally- and regionally-appropriate cell types in response to microenvironmental cues, and non-disruptive, non-tumorigenic interspersion with host progenitors and their progeny. Human NSCs are capable of expressing foreign transgenes in vivo in these disseminated locations. As such, these cells find use in the treatment of a variety of conditions, including traumatic injury to the spinal cord, brain, and peripheral nervous system; treatment of degenerative disorders including Alzheimer's disease, Huntington's disease, Parkinson's disease; affective disorders including major depression; stroke; and the like. By synaptogenesis enhancers, the functional connections of the neurons are enhanced, providing for an improved clinical outcome.

The findings of the present invention also provide a means to modulate axonal and/or dendritic growth by means of new synaptogenesis and to improve function after CNS damage. The induction of axonal and/or dendritic growth will promote functional improvement after injury. The amount of increase in axonal and/or dendritic growth may comprise at least a measurable increase relative to a control lacking such treatment, for example at least a 10% increase, at least a 20% increase, at least a 50% increase, or more. In some embodiments, the axonal and/or dendritic growth may be increased at least about any of 10%, 20%, 30%, 40%, 50%, or 60%. In some embodiments, the axonal and/or dendritic growth is axon growth. In some embodiments, the axonal and/or dendritic growth is dendritic growth.

In some embodiments of any of the methods described herein, the individual or subject may have suffered axonal and/or dendritic degeneration as a result of a spinal cord injury. In some embodiments, the individual or subject has suffered axonal and/or dendritic degeneration as a result of Alzheimer's disease, Parkinson's disease, ALS, multiple sclerosis, Huntington disease, Down syndrome, spinal mascular atrophy, spinal cord injury, Myofiber atrophy, denervation atrophy, or glaucoma. In some embodiments, the individual or subject has suffered a macular degeneration, a hearing loss, a diabetic neuropathy, or a chemotherapy induced neuropathy. In some embodiments, the individual or subject has suffered axonal and/or dendritic degeneration as a result of a psychiatric disorder selected from the group consisting of acute stress disorder, aggression, agoraphobia, autism, depression, dissociative amnesia, anorexia nervosa, bipolar disorder, body dysmorphic disorder, brief psychotic disorder, bulimia nervosa, conversion disorder, cyclothymic disorder, delusional disorder, depersonalization disorder, dissociative identity disorder (DID), dysparenunia, dysthymic disorder, male erectile disorder, generalized anxiety disorder, impotence, pain disorder, phobias, posttraumatic stress disorder, schizoaffective disorder, schizophrenia, schizophreniform, shared psychotic disorder, and substance abuse. In some embodiments, the individual or subject has suffered axonal and/or dendritic degeneration as a result of a viral infection selected from the group consisting of poliomyelitis virus, West Nile Virus, and Human immunodeficiency virus (HIV). In some embodiments, the individual or subject has suffered axonal and/or dendritic degeneration as a result of a Prion disease selected from the group consisting of kuru, Creutzfeldt-Jakob disease, variant Creutzfeldt-Jakob disease, Gerstmann-Sträussler-Scheinker syndrome, and fatal familial insomnia.

Gene Delivery

One approach for inducing synaptogenesis involves gene therapy. In such methods, sequences encoding an antibody or a polypeptide described herein are introduced into the central nervous system, and expressed, as a means of providing agonist activity to the targeted cells. To genetically modify neurons that are protected by the blood brain barrier (BBB), two general categories of approaches have been used. In one type of approach, cells are genetically altered, outside the body, and then transplanted somewhere in the CNS, usually in an area inside the BBB. In the other type of approach, genetic “vectors” are injected directly into one or more regions in the CNS, to genetically alter cells that are normally protected by the BBB. It should be noted that the terms “transfect” and “transform” are used interchangeably herein. Both terms refer to a process which introduces a foreign gene (also called an “exogenous” gene) into one or more preexisting cells, in a manner which causes the foreign gene(s) to be expressed to form corresponding polypeptides.

A preferred approach aims to introduce into the CNS a source of a desirable polypeptide, by genetically engineering cells within the CNS. This has been achieved by directly injecting a genetic vector into the CNS, to introduce foreign genes into CNS neurons “in situ” (i.e., neurons which remain in their normal position, inside a patient's brain or spinal cord, throughout the entire genetic transfection or transformation procedure).

Useful vectors include viral vectors, which make use of the lipid envelope or surface shell (also known as the capsid) of a virus. These vectors emulate and use a virus's natural ability to (i) bind to one or more particular surface proteins on certain types of cells, and then (ii) inject the virus's DNA or RNA into the cell. In this manner, viral vectors can deliver and transport a genetically engineered strand of DNA or RNA through the outer membranes of target cells, and into the cells cytoplasm. Gene transfers into CNS neurons have been reported using such vectors derived from herpes simplex viruses (e.g., European Patent 453242, Breakfield et al 1996), adenoviruses (La Salle et al 1993), and adeno-associated viruses (Kaplitt et al 1997).

Non-viral vectors typically contain the transcriptional regulatory elements necessary for expression of the desired gene, and may include an origin of replication, selectable markers and the like, as known in the art. The non-viral genetic vector is then created by adding, to a gene expression construct, selected agents that can aid entry of the gene construct into target cells. Several commonly-used agents include cationic lipids, positively charged molecules such as polylysine or polyethylenimine, and/or ligands that bind to receptors expressed on the surface of the target cell. For the purpose of this discussion, the DNA-adenovirus conjugates described by Curiel (1997) are regarded as non-viral vectors, because the adenovirus capsid protein is added to the gene expression construct to aid the efficient entry of the gene expression construct into the target cell.

In cationic gene vectors, DNA strands are negatively charged, and cell surfaces are also negatively charged. Therefore, a positively-charged agent can help draw them together, and facilitate the entry of the DNA into a target cell. Examples of positively-charged transfection agents include polylysine, polyethylenimine (PEI), and various cationic lipids. The basic procedures for preparing genetic vectors using cationic agents are similar. A solution of the cationic agent (polylysine, PEI, or a cationic lipid preparation) is added to an aqueous solution containing DNA (negatively charged) in an appropriate ratio. The positive and negatively charged components will attract each other, associate, condense, and form molecular complexes. If prepared in the appropriate ratio, the resulting complexes will have some positive charge, which will aid attachment and entry into the negatively charged surface of the target cell. The use of liposomes to deliver foreign genes into sensory neurons is described in various articles such as Sahenk, 1993. The use of PEI, polylysine, and other cationic agents is described in articles such as Li,2000 and Nabel, 1997.

An alternative strategy for introducing DNA into target cells is to associate the DNA with a molecule that normally enters the cell. This approach was demonstrated in liver cells in U.S. Pat. No. 5,166,320 (Wu et al 1992). An advantage of this approach is that DNA delivery can be targeted to a particular type of cell, by associating the DNA with a molecule that is selectively taken up by that type of target cell. A limited number of molecules are known to undergo receptor mediated endocytosis in neurons. Known agents that bind to neuronal receptors and trigger endocytosis, causing them to enter the neurons, include (i) the non-toxic fragment C of tetanus toxin (e.g., Knight et al 1999); (ii) various lectins derived from plants, such as barley lectin (Horowitz et al 1999) and wheat germ agglutinin lectin (Yoshihara et al 1999); and, (iii) certain neurotrophic factors (e.g., Barde et al 1991). At least some of these endocytotic agents undergo “retrograde” axonal transport within neuron. The term “retrograde”, in this context, means that these molecules are actively transported, by cellular processes, from the extremities (or “terminals”) of a neuron, along an axon or dendrite, toward and into the main body of the cell, where the nucleus is located. This direction of movement is called “retrograde”, because it runs in the opposite direction of the normal outward (“anterograde”) movement of most metabolites inside the cell (including proteins synthesized in the cell body, neurotransmitters synthesized by those proteins, etc.).

Methods of Administration and Dosages

Administration of the antibodies and polypeptides described herein can be achieved in various ways, including oral, buccal, rectal, parenteral, intraperitoneal, intradermal, transdermal, intrathecal, nasal, intranasal, topical, intravenous, intraarterial, intramuscular, subcutaneous, subdermal, intracranial, ophthalmic (e.g., topical, injection (e.g., subconjunctival, subtenon, intravitreal, etc.), or implantation), or intrathecal administration. The antibodies or polypeptides may be systemic after administration or may be localized by the use of regional administration, intramural administration, or use of an implant that acts to retain the active dose at the site of implantation.

The antibodies or polypeptides of the invention may be administered using any medically appropriate procedure, e.g. intravascular (intravenous, intraarterial, intracapillary) administration, injection into the cerebrospinal fluid, intracavity or direct injection in the brain. Intrathecal administration maybe carried out through the use of an Ommaya reservoir, in accordance with known techniques. (F. Balis et al., Am J. Pediatr. Hematol. Oncol. 11, 74, 76 (1989).

One strategy for drug delivery through the blood brain barrier (BBB) entails disruption of the BBB, either by osmotic means such as mannitol or leukotrienes, or biochemically by the use of vasoactive substances such as bradykinin. The potential for using BBB opening to target specific agents is also an option. A BBB disrupting agent can be co-administered with the therapeutic antibodies or polypeptides of the invention when the compositions are administered by intravascular injection. Other strategies to go through the BBB may entail the use of endogenous transport systems, including carrier-mediated transporters such as glucose and amino acid carriers, receptor-mediated transcytosis for insulin or transferrin, and active efflux transporters such as p-glycoprotein. Active transport moieties may also be conjugated to the therapeutic or imaging compounds for use in the invention to facilitate transport across the epithelial wall of the blood vessel. Alternatively, drug delivery behind the BBB is by intrathecal delivery of therapeutics or imaging agents directly to the cranium, as through an Ommaya reservoir.

Where the antibodies or polypeptides are locally administered in the brain, one method for administration of the therapeutic compositions of the invention is by deposition into or near the site by any suitable technique, such as by direct injection (aided by stereotaxic positioning of an injection syringe, if necessary) or by placing the tip of an Ommaya reservoir into a cavity, or cyst, for administration. Alternatively, a convection-enhanced delivery catheter may be implanted directly into the site, into a natural or surgically created cyst, or into the normal brain mass. Such convection-enhanced pharmaceutical composition delivery devices greatly improve the diffusion of the composition throughout the brain mass. The implanted catheters of these delivery devices utilize high-flow microinfusion (with flow rates in the range of about 0.5 to 15.0 μl/minute), rather than diffusive flow, to deliver the therapeutic composition to the brain and/or tumor mass. Such devices are described in U.S. Pat. No. 5,720,720, incorporated fully herein by reference.

In some embodiments of the methods of treatment and methods of administration described herein, the methods include administering an effective amount of an antibody or polypeptide to promote synapse formation. In some embodiments of the methods of treatment and methods of administration described herein, the methods include administering an effective amount of an antibody or polypeptide to promote axonal and/or growth. In some embodiments, the antibody is an agonist.

In some embodiments of the methods of treatment and methods of administration described herein, the methods comprise administering one or more antibodies or polypeptide described herein. In some embodiments, the methods include (a) administering an effective amount of a first antibody to promote axonal and/or dendritic growth and (b) administering an effective amount of a second antibody to promote synapse formation. In some embodiments, the first antibody and second antibody are administered sequentially. In some embodiments, the first antibody and second antibody are administered separately. In some embodiments, the first antibody is administered less than about any of 1, 2, 3, 4, 5, 6, 7, 8, 9, or 10 days prior to the second antibody. In some embodiments, the second antibody is administered less than about any of 1, 3, 6, 9, 12, 18, 24, hours after first antibody.

The methods of treatment and methods of administration described herein including antibodies or polypeptide of the present invention, are administered at a dosage that induces synaptogenesis and/or axon growth while minimizing any side-effects. It is contemplated that the antibodies or polypeptides will be obtained and used under the guidance of a physician for in vivo use. The dosage of the therapeutic formulation will vary widely, depending upon the nature of the disease, the frequency of administration, the manner of administration, the clearance of the antibodies or polypeptides from the host, and the like.

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

The effective amount of a therapeutic composition described herein to be given to a particular patient will depend on a variety of factors, several of which will be different from patient to patient. Dosage of the antibodies or polypeptides will depend on the treatment, route of administration, the nature of the therapeutics, sensitivity of the patient to the therapeutics, etc. Utilizing LD₅₀ animal data, and other information, a clinician can determine the maximum safe dose for an individual, depending on the route of administration. Utilizing ordinary skill, the competent clinician will be able to optimize the dosage of a particular therapeutic composition in the course of routine clinical trials. The compositions can be administered to the subject in a series of more than one administration. For therapeutic compositions, regular periodic administration will sometimes be required, or may be desirable. Therapeutic regimens will vary with the antibodies or the polypeptides, e.g. some antibodies or polypeptides may be taken for extended periods of time on a daily or semi-daily basis, while more selective antibodies or polypeptides may be administered for more defined time courses, e.g. one, two three or more days, one or more weeks, one or more months, etc., taken daily, semi-daily, semi-weekly, weekly, etc.

Pharmaceutically Acceptable Compositions and Formulations

The invention also provides pharmaceutical compositions comprising an antibody or a polypeptide that specifically binds to an epitope in a VWF-A domain of a calcium channel subunit α2δ and induces synaptogenesis and a pharmaceutically acceptable carrier. The antibodies or polypeptides can be incorporated into a variety of formulations for therapeutic administration by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The compositions can also include any of a variety of stabilizing agents, such as an antioxidant for example. The pharmaceutical composition of an antibody can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The antibodies of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Formulations may be optimized for retention and stabilization in the brain. When the antibody is administered into the cranial compartment, it is desirable for the antibody to be retained in the compartment, and not to diffuse or otherwise cross the blood brain barrier. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the antibody in a biodegradable or bioerodible implant. The rate of release of the therapeutically active antibody is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the subject invention. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

### Articles of Manufacture and Kits

The invention provides articles of manufacture comprising the compositions, formulations, and unit dosages described herein in suitable packaging for use in the methods of treatment and methods of administration described herein. The compositions of the invention comprise an antibody or a polypeptide that specifically binds to an epitope in a VWF-A domain of a calcium channel subunit α2δ and induces synaptogenesis. Suitable packaging for compositions described herein are known in the art, and include, for example, vials (such as sealed vials), vessels (such as sealed vessels), ampules, bottles, jars, flexible packaging (e.g., sealed Mylar or plastic bags), and the like. These articles of manufacture may further be sterilized and/or sealed.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

Instructions supplied in the kits of the invention are typically written instructions on a label or package insert (e.g., a paper sheet included in the kit), but machine-readable instructions (e.g., instructions carried on a magnetic or optical storage disk) are also acceptable. The instructions relating to the use of the nanoparticle compositions generally include information as to dosage, dosing schedule, and route of administration for the intended treatment. The kit may further comprise a description of selecting an individual suitable or treatment.

The present invention further provides kits comprising compositions (or unit dosages forms and/or articles of manufacture) described herein and may further comprise instruction(s) on methods of using the composition, such as for inducing synaptogenesis in an individual. In some embodiments, the kit of the invention comprises the packaging described above. In other embodiments, the kit of the invention comprises the packaging described above and a second packaging comprising a buffer. It may further include other materials desirable from a commercial and user standpoint, including other buffers, diluents, filters, needles, syringes, and package inserts with instructions for performing any methods described herein.

### EXAMPLES

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.), but some experimental errors and deviations should be allowed for. Unless otherwise indicated, molecular weight is average molecular weight and temperature is in degrees centigrade.

### Example 1

### Generation of Antibodies that Bind to VWF-A Domain of Calcium Channel α2δ1 and α2δ2 Subunit

Peptides spanning regions which are largely conserved between the VWF-A domains of human α2δ1 and α2δ2 subunits were used to generate mouse hybridomas. Two peptides, MZp 109 having the amino acid sequence of RIVRVFTFSVGQHNYDVTPLQWMACANKGYYFEIPSIGAIRINTQEKK (SEQ ID NO:5) from human α2δ2 VWF-A domain, and MZp110 having the amino acid sequence of KKVRVRFSVGQHNYERGPIQWMACENKGYYYEIPSIGAIRINTQER (SEQ ID NO:6) from human α2δ1 VWF-A domain were used for immunization to generate mouse monoclonal antibodies.

ELISA Conditions

For screening and testing: 0.5 μg/well of MZp109 and MZp110 antigens were coated onto separate plates in dH₂O at 50 μL/well and dried down overnight at 37° C. For testing by antibody trapping assay: 1/10000 Goat anti-mouse IgG/IgM trapping antibody (Pierce cat# 31182) was coated onto plate in carbonate coating buffer (pH 9.6) at 1004/well incubated overnight at 4° C. For testing on negative control antigen: 0.41 μg/well HT (human transferrin) antigens was coated onto plate in dH₂O at 50 μL/well and dried down overnight at 37° C. Blocking: Plates blocked with 3% skim milk powder in PBS (pH 7.4) at 100 μL/well were incubated for 1 hour at room temperature. Primary antibody: Mouse anti-MZp109/110-RP hybridoma tissue culture supernatant and mouse monoclonal controls were added at 1004 neat per well for screening and testing. Mouse anti-MZp 109 or anti-MZp 110 immune serum and mouse pre-immune serum were diluted at 1/400 in SP2/0 tissue culture supernatant and were then added at 100 μL/well for screening and testing. The anti-MZp109 or anti-MZp110 immune serum and pre-immune serum were incubated for 1 hour at 37° C. with shaking for both the screening and testing. Secondary antibody: 1/25000 Goat anti-mouse IgG Fc HRP conjugated antibody (Pierce cat#31439) was used in screening and testing. Secondary antibody was diluted in PBS-Tween, added at 100 μL/well, and incubated for 1 hour at 37° C. with shaking. Substrate: TMB buffer (BioFx cat# TMBW-1000-01) was added at 504 per well and incubated in the dark at room temperature. Reactions for screening and testing was stopped with 504 of 1M HCl per well after 10 minutes and was read at OD₄₅₀ nm.

A summary of ELISA analysis of various hybridoma clones expressing mouse monoclonal antibodies against peptides MZp 109 and MZp 110 is listed in Table 1.

TABLE 1
ELIZA analysis of mouse hybridoma clones
against peptides MZp109 and MZ-110

| | | | | Final | |
|---|---|---|---|---|---|
| | | | | Stability | |
| | Screening | Screening | Testing | Test by | |
| | on | on | on | Trapping | |
| Clone | MZp109 | MZp110 | HT | Assay | Isotype |
| 1F5 | 1.001 | 0.643 | 0.089 | 1.559 | IgG |
| 1G6 | 2.841 | 2.247 | 0.108 | 1.610 | IgG |
| 2B9 | 2.804 | 2.057 | 0.098 | 1.589 | IgG |
| 2E4 | 2.102 | 1.489 | 0.084 | 1.737 | IgG |
| 2E9 | 1.628 | 1.124 | 0.073 | 1.594 | IgG |
| 2G9 | 2.786 | 2.229 | 0.090 | 2.352 | IgG |
| 2G10 | 2.587 | 1.632 | 0.080 | 0.975 | IgG |
| 3A7 | 1.390 | 1.496 | 0.077 | 0.655 | IgG |
| 3B4 | 3.103 | 2.132 | 0.084 | 1.556 | IgG |
| 3F9 | 1.788 | 1.049 | 0.087 | 1.871 | IgG |
| 4B2 | 1.655 | 1.653 | 0.082 | 1.329 | IgG |
| 5A5 | 2.628 | 1.879 | 0.089 | 1.525 | IgG |
| 5H3 | 2.672 | 1.985 | 0.079 | 1.267 | IgG |
| 5H4 | 2.182 | 1.964 | 0.081 | 1.487 | IgG |
| 5H7 | 2.222 | 1.854 | 0.083 | 1.506 | IgG |

### Example 2

### Characterization of Monoclonal Antibodies Against VWF-A Domain of Calcium Channel α2δ1 and α2δ2 Subunit

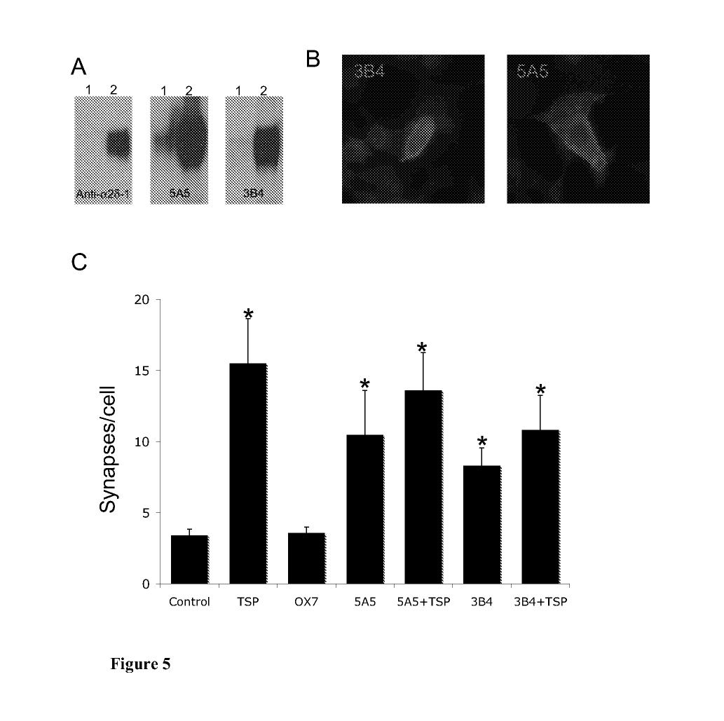

Monoclonal antibodies from various clones were further characterized by Western blot using membranes from a cell line of human embryonic kidney origin (HEK293) that over-expressed the rat α2δ1 protein (Table 2 and FIG. 5A) and by their ability to immunostain the cell surface of the HEK293 cells which over-expressed the rat α2δ1 protein (FIG. 5B). The 5A5 antibody was also characterized by immunostaining brain sections utilizing the array tomography technique as recently described (Micheva et al 2007). Ultra-thin sections of rat cortex or mouse LGN (lateral geniculate nucleus) were immunolabelled with antibody 3B4 or 5A5 against α2δ, and an antibody against the presynaptic marker synapsin. The anti-α2δ antibodies (5A5) gave a very punctate staining pattern. Some of these puncta were localized to synapses identified as juxtapositioned pre and post-synaptic puncta. In addition, some α2δ puncta co-localized exclusively with pre or post-synaptic puncta. These results show that α2δ protein is localized to synapses and is closely associated with pre and post-synaptic proteins.

TABLE 2
Binding of antibodies to cell membranes
containing the rat α2δ1 protein

| | Blot |
|---|---|
| Antibody | results |
| 1F5 | − |
| 1G6 | ++ |
| 2B9 | + |
| 2E4 | − |
| 2E9 | − |
| 2G9 | ++ |
| 2G10 | − |
| 3A7 | ND |
| 3B4 | +++++ |
| 3F9 | + |
| 4B2 | − |
| 5A5 | +++++ |
| 5H3 | +++ |
| 5H4 | − |
| 5H7 | − |

The antibodies from clone 5A5 and 3B4 were further tested for their ability to modulate the function of α2δ1 in synapse formation. Retinal ganglion neurons (RGC) were cultured with these antibodies in the presence or absence of the synapse inducing protein Thrombospondin (TSP) and synapse number was analyzed. Both 5A5 and 3B4 antibody were found to induce synapse formation in the absence of TSP, while a control antibody (0×7) against another RGC surface receptor, Thy1, did not. The synaptogenic effect of 5A5 and 3B4 were not additive with that of TSP (FIG. 5C). This result shows that antibody binding to the VWF-A domain can mimic the synaptogenic function of TSP and suggests that the interaction of TSP with the VWF-A domain of α2δ1 is important for the initiation of synapse formation. The fact that antibodies directed against the domain of α2δ1 mimic the synaptogenic function of TSP also suggests a binding-induced activation of α2δ1 in synapse formation. For synapse quantification of RGC cultures, cells were fixed for 7 minutes with 4% paraformaldehyde (PFA), washed three times in phosphate-buffered saline (PBS), and blocked in 100 μl of a blocking buffer containing 50% normal goat serum and 0.1% Triton X-100 for 30 minutes. After blocking, coverslips were washed three times in PBS, and 100 μL of primary antibody solution was added to each coverslip, consisting of rabbit anti-synaptotagmin (1:750, cytosolic domain, Synaptic Systems) and mouse anti-PSD-95 (1:750, 6G6-1C9 clone, Affinity Bio Reagents). Coverslips were incubated overnight at 4° C., washed three times in PBS, and incubated with 100 μl of Alexa-594 conjugated goat anti-rabbit and Alexa-488 conjugated goat anti-mouse (Invitrogen) diluted 1:1000 in antibody buffer. Following a 2-hour incubation, coverslips were washed 3-4 times in PBS and mounted in Vectashield mounting medium with DAPI (Vector Laboratories Inc) on glass slides (VWR Scientific). Secondary-only controls were routinely performed and revealed no significant background staining.

### Example 3

### Testing Antibody 5A5 and 3B4 in Animal Disease Models

### Tests in Traumatic Closed Head Injury Model

Traumatic brain injury is the major cause of mortality and morbidity in the young age group (15-40 yr), with short and long term consequences. It accounts for an estimated 2 million new cases per year (USA). It is also a risk factor for the development of devastating neurodegenerative diseases (Alzheimer's and Parkinson diseases) which develops years later. To date, there is no effective drug therapy to these conditions, and patients surviving trauma often need special care for the rest of their lives, creating an enormous financial burden. There is a need for drugs that could inhibit the harmful consequence or augment and protect nerve cells from the injury or, could enhance regenerative and recovery pathways.

Animals: Male mice weighing 25-35 g are used in this study. They are housed in groups of 6 per cage, in a 12 h:12 h light:dark reversed light cycle. Food and water are provided ad libitum. The animals are divided into 4 experimental groups, and assessed at different times after closed head injury (CHI).

Trauma model: Trauma is induced under isoflurane anesthesia, which is confirmed by testing loss of pupillary and corneal reflexes. A longitudinal incision in the skin covering the skull is performed, and the skin is retracted, to expose the skull bone. The head is manually fixed at the bottom plane of the impact device, using a modification of the weight-drop device previously described (Chen et al., J. Neurotrauma 13:557-568, 1996).

Neurological severity score: Motor function and reflexes of the injured rats are evaluated at different times after CHI using a neurological severity score (NSS, see below and Tsenter et al., J. Neurotrauma 25; 324-333, 2008), which emphasizes the motor functions. One point is awarded for the lack of a tested reflex or for the inability to perform the tasks. The maximal score that could be reached is 10 points. The differences between NSS at 1 h (the earliest possible time of testing) and those at later times after the impact reflect the spontaneous (or drug-induced) recovery, and is termed acute ΔNSS. This is a useful parameter for the evaluation of the effects of drugs in the CHI model.

After evaluation the initial NSS (1 h) the drugs is injected (no earlier than 1 h post injury) and their NSS will be evaluated at 24 h. Some mice will be sacrificed at 24 h for evaluation of edema (see below) and NSS of the remaining will be assessed at 3 and 7 d and up to 30 days. NSS and ΔNSS in the four groups are compared.

Drug Treatment.

Selection of the optimal dose: The effect of the drug on naïve, non-injure mice is examined. Then, 60 mice which underwent CHI (n=15/group) are treated 1 h after injury and then periodically until the experiment is terminated, with either the vehicle or with several doses of antibody 5A5 or 3B4. NSS is assessed from 24 hrs every 1-2 day in the first week, and than, once a week, up to a month.

Selection of the optimal time of administration: In another set of experiments, the dose selected in the first set is injected at 1, 2, 4, 8, 24, 48 hrs, or 14 days post CHI and then periodically until experiment is terminated. Assessment of NSS is performed as above.

Selection of the optimal treatment regimen: To examine whether a single, or repeated dosing is effective, 4 groups of mice are treated with the drug either as a single dose post injury based on the result of the experiment described above, double or triple (administered at the most effective time), and another dose 24 hrs or 24+48 hr later.

The protocol described above is designed to provide evidence for the beneficial effect of the tested compound. If positive, a second phase of the study examines effect on physiological (edema, blood brain barrier integrity, necrosis etc.) or neurochemical pathways (inflammation, apoptosis). Table 3 below shows tasks measured for determining neurological severity scores.

TABLE 3
Neurological Severity Score for head injured mice

| TASK | NSS |
|---|---|
| Presence of mono- or hemiparesis | 1 |
| Inability to walk on a 3 cm wide beam | 1 |
| Inability to walk on a 2 cm wide beam | 1 |
| Inability to walk on a 1 cm wide beam | 1 |
| Inability to balance on a 1 cm wide beam | 1 |
| Inability to balance on a round stick (0.5 cm wide) | 1 |
| Failure to exit a 30 cm diameter circle (for 2 min) | 1 |
| Inability to walk straight | 1 |
| Loss of startle behavior | 1 |
| Loss of seeking behavior | 1 |
| Maximum Total | 10 |

One point is awarded for failure to perform a task. NSS at 1 h in the range of 8-10: severe CHI

### Tests in a Stroke MCA Occlusion (MCAD) in Rats

Animals: 48 male Sprague Dawley Rats, 300-400 g, Charles River Laboratories, (to arrive 7-10 days before surgery at 250-275 g). The following tests for antibody 5A5 and 3B4 are conducted:

Antibody, Dose 1 (0.1 mg/kg), i.p., 1× or 2× per week, starting 1 hours after MCAO (N=12)

Antibody, Dose 2 (1 mg/kg), i.p., 1× or 2× per week, starting 24 hours after MCAO (N=12)

Antibody, Dose 3 (10 mg/kg), i.p., 1× or 2× per week, starting 48 hours after MCAO (N=12)

Vehicle, i.p., 2× per week, starting 1 hours after MCAO (N=12)

The nomenclature for the days of the study is as follows: Day 0 is the day of the MCAO, and the days following are numbered consecutively (Day 1, Day 2, Day 3, etc.) Day −1 represents the day prior to the MCA.

Grouping details: The amount of time needed for some procedures in this study necessitated breaking up the 4 treatment groups (as listed above), into 8 working groups (as written in the schedule). Six animals receive stroke surgery per day. If an animal dies during the 8-day surgical period of the study, it is replaced by a spare. If not, the animal is not replaced. Most animal deaths (<5% overall) occur in the immediate post-op to 7 day period.

Anesthesia: Anesthesia is induced in an induction chamber with 2-3% isoflurane in N₂O:O₂ (2:1), and maintained with 1-1.5% isoflurane via face mask. Adequate depth of anesthesia is assessed by lack of withdrawal to hindlimb pinch and loss of eyeblink reflex. Once anesthetized, animals receive cefazolin sodium (40 mg/kg, i.p.) and buprenorphine (0.1 mg/kg, s.c.). A veterinary ophthalmic ointment, Lacrilube, is applied to the eyes. Temperature is kept at 37.0±1° C.

Surgical Procedure: A small focal stroke (infarct) is made on the right side of the surface of the brain (cerebral cortex) by middle cerebral artery occlusion (MCAO). The right side of the head is shaved with electric clippers (patch of approx 3×5 cm between eye and ear). The region is carefully cleaned with septisol. Using aseptic technique, an incision is made midway between the eye and eardrum canal. The temporalis muscle is isolated, bisected, and reflected. A small window of bone is removed via drill and rongeurs (subtemporal craniectomy) to expose the MCA. Care is taken not to remove the zygomatic arch or to transect the facial nerve that would impair the ability of the animal to chew after surgery. Using a dissecting microscope, the dura is incised, and the MCA is electrocoagulated from just proximal to the olfactory tract to the inferior cerebral vein (taking care not to rupture this vein), using microbipolar electrocauterization. The MCA is then transected. The temporalis muscle is then repositioned, and the incision is closed subcutaneously with sutures. The skin incision is closed with surgical staples (2-3 required). Throughout the procedure, body temperature is maintained at 37.0°±1° C., using a self-regulating heating pad connected to a rectal thermometer.

Post-Operative Monitoring: Following surgery, animals remain on a heating pad until they awaken from anesthesia. They are then returned to clean home cages. They are observed frequently on the day of MCAO surgery (Day 0) and at least once daily thereafter.

Handling, surgery, injections and blood collection timetable: The animals are housed 2-3 per cage before and after surgery, unless severe aggression is displayed, or death of cage mate(s). The animals are housed in the case for 7 days before the surgery. Cefazolin Sodium is administered i.p. (40 mg/kg), and Buprenorphine is administered s.c. (0.1 mg/kg), right before surgery. Middle cerebral artery occlusion (MCAO) is conducted on Day 0 (modified Tamura model). Antibody 5A5, 3B4 or vehicle is administered i.p. to the animals one or two times per week starting 1 hour, 24 hours, or 48 hours after MCAO.

On days when both drug administration and behavioral tests are conducted, behavioral tests are performed before drug administration. Behavioral evaluations are done by investigators blinded to treatment assignment.

Limb Placing: Evaluations are conducted on Day −1 (pre-operation), Day 1, Day 3, Day 7, Day 14, Day 21, Day 28. The limb placing tests are divided into both forelimb and hindlimb tests. For the forelimb-placing test, the investigator holds the rat close to a tabletop and scores the rat's ability to place the forelimb on the tabletop in response to whisker, visual, tactile, or proprioceptive stimulation. Similarly, for the hindlimb placing test, the investigator assesses the rat's ability to place the hindlimb on the tabletop in response to tactile and proprioceptive stimulation. Separate sub-scores are obtained for each mode of sensory input and added to give total scores (for the forelimb placing test: 0=normal, 12=maximally impaired; for the hindlimb placing test: 0=normal; 6=maximally impaired). Scores are given in half-point increments (see below). Typically, there is a slow and steady recovery of limb placing behavior during the first month after stroke.

Forelimb placing test (0-12):

whisker placing (0-2);

visual placing (forward (0-2), sideways (0-2))

tactile placing (dorsal (0-2), lateral (0-2))

proprioceptive placing (0-2).

Hindlimb placing test (0-6):

tactile placing (dorsal (0-2), lateral (0-2))

proprioceptive placing (0-2).

For each subtest, animals are scored as followed:

0=immediate response

0.5=response within 2 seconds

1.0=response of 2-3 seconds

1.5=response of >3 seconds

2.0=no response

Body Swing: Evaluations are conducted on Day −1 (pre-operation), Day 1, Day 3, Day 7, Day 14, Day 21, Day 28. The rat is held approximately one (1) inch from the base of its tail. It is then elevated to an inch above a surface of a table. The rat is held in the vertical axis, defined as no more than 10° to either the left or the right side. A swing is recorded whenever the rat moves its head out of the vertical axis to either side. Before attempting another swing, the rat must return to the vertical position for the next swing to be counted. Thirty (30) total swings are counted. A normal rat typically has an equal number of swings to either side. Following focal ischemia, the rat tends to swing to the contralateral side (left side in this case). Body swing scores are expressed as a percentage of rightward over total swings. There is a spontaneous partial recovery of body swing scores (toward 50%) during the first month after stroke.

Sacrifice: On day 28 after MCAO, rats are anesthetized deeply with ketamine/xylazine (100 mg/kg ketamine, 10 mg/kg xylazine, i.p.) and perfused transcardially with normal saline (with heparin 2 unit/ml) followed by 10% formalin. Brains are processed for infarct volume measurement (H&E staining).

Infarct measurement: Seven sections (+4.7, +2.7, +0.7, −1.3, −3.3, −5.3 and −7.3, compared to bregma respectively) from each brain are photographed by a digital camera, and the infarct area on each slice is determined by NIH Image (Image J) using the “indirect method” (area of the intact contralateral [left] hemisphere—area of intact regions of the ipsilateral [right] hemisphere) to correct for brain edema. Infarct areas are then summed among slices and multiplied by slice thickness to give total infarct volume, which is expressed as a percentage of intact contralateral hemispheric volume.

### Tests in Amyotrophic Lateral Sclerosis (ALS) Models

Amyotrophic lateral sclerosis (ALS) usually attacks both upper and lower motor neurons and causes degeneration throughout the brain and spinal cord. Early symptoms in 50% percent of affected people are a painless weakness in a hand, foot, arm, or leg. Other early symptoms include speech-swallowing and difficulty walking. ALS most commonly strikes people between 40 and 70 years old, affecting as many as 30,000 Americans at any given time. Like other neuromuscular diseases, ALS is complex, but spontaneous mouse mutants are useful to identify the responsible genes and biochemical pathways.

The following mouse models of the human ALS may be used to test the effect of antibody 5A5 and 3B4. See Gurney et al., Science 264:1772-5 (1994); Cook et al., Mamm. Genome 6:187-91; Cox et al., Neuron 21:1327-37 (1998); Iga et al., PNAS 106:18809-18814 (2009).

B6SJL-Tg(SOD1*G93A)1Gur/J are transgenic mice carrying a high copy number of a mutant allele human SOD1 containing the Gly93→Ala (G93A) substitution (often referred to as G1H). Due to loss of motor neurons in the spinal cord, hemizygous transgenics become paralyzed in one or more limbs and die by the time they are four to five months old.

B6SJL-Tg(SOD1*G93A)^{d1}1Gura are transgenic mice carrying a variant of the human superoxide dismutase-1 gene (glycine to alanine at position 93). They have a lower transgene copy number and develops symptoms later than mutants of the original strain mentioned above (B6SJL-Tg(SOD1-G93A)1Gur/J). They become paralyzed in one or more limbs when they are around six to seven months old and die four to six weeks later.

B6.Cg-Tg(SOD1*G93A)1Gur/J are transgenic mice carrying a high copy number of the mutant allele human SOD1 containing the Gly93→Ala (G93A) substitution. Hemizygous transgenic mice become paralyzed in one or more limbs and have a life span of approximately 19-23 weeks. Paralysis is due to loss of motor neurons from the spinal cord.

B6.BKS-Ighmbp2^{nmd-2J}/J have the phenotypes of nmd mutants that are very similar to those in human ALS, SMA, and SMARD1. The responsible mutation is a defect in the immunoglobulin S-mu binding protein 2 (Ighmbp2) gene. Mutants are easily identifiable by the time they are two weeks old. Paralysis usually begins in the hindlimbs and progresses to the forelimbs. Balance is not affected. Most homozygotes die by the time they are four weeks old. Heterozygotes are phenotypically and histologically normal.

TDP-43—is a mouse model having a point mutation in the gene for a protein called TDP-43 which is linked to inherited forms of ALS. The mice have damage to both upper motor neurons, developed a characteristic “swimming” gait, where they were unable to hold their body off the ground and die prematurely.

Experimental protocol: All transgenic mice are genotyped by PCR amplification of DNA extracted from the tails to identify the SOD-1, of NMD or TDP-43 mutation. Mice are randomly divided into vehicle, or drug groups, and are matched for littermates. Drug treatments are initiated at various times after birth and continued until the end stage with 6-15 animals in each group. Drug (antibody 5A5 or 3B4) is injected intraperitoneally (i.p.) twice weekly. The vehicle control group receives injections of the same volume of saline. Mice are maintained on a 12-hr light/dark cycle and the behavior tests are performed during the light period. The onset and severity of motor dysfunction as well as survival time are determined.

Behavioral assessment and analysis: Starting from 12 weeks of age and until death, various behavioral tests are routinely performed to assess the effects of drugs on the onset and extent of neurological deficits. All tests are performed by an investigator who is blind to the experimental conditions of the animals.

Rotarod performance test: After training sessions are conducted to acclimate the mice to the rotarod apparatus (Columbus Instruments, Columbus, Ohio, USA), motor coordination is assessed by measuring the length of time at which the mice remain on the rotating rod (16 rpm) as described (Azzouz et al., Nature 429:413-417, 2004). Three trials are given to each animal and the longest retention time is used as the measure of competence at this task. The evaluation scores are: grade 0, >180 sec; grade 1, 60-180 sec; grade 2, <60 sec; grade 3, falling off the rod before rotation.

Postural reflex test: This is conducted essentially as described (Bederson et al., Stroke 17:472-476, 1986) to examine the strength of the forelimbs. The deficits were defined and scored as follows: grade 0, no evidence of paralysis; grade 1, forelimb flexion upon tail suspension; grade 2, decreased resistance to lateral push (and forelimb flexion) without circling; grade 3, same as grade 2 but with circling; grade 4, unable to walk but maintaining upright body position; grade 5, complete paralysis.

Balance beam test: This is performed essentially as described (Feeney et al., Science 217:855-857, 1982) to measure body strength and equilibrium. The deficits are scored as follows: grade 0, able to lift itself onto the beam and walk without falling off; grade 1, same as grade 0 but with less than 50% chance of falling off; grade 2, same as grade 1 with more than 50% chance of falling off; grade 3, able to lift itself onto the beam with assistance but unable to move forward; grade 4, unable to lift onto the beam but able to stay in position; grade 5, falling off the beam instantaneously after placement.

Screen test: This test serves as an indicator of general muscle strength (Combs and D'Alecy, Stroke 18:503-511, 1987). The animal is placed on a horizontally positioned screen with grids. The screen is then rotated to the vertical position. The deficit scores are: grade 0, grasping the screen with forepaws for more than 5 sec; grade 1, temporarily holding the screen without falling off; grade 2, same as grade 1 but falling off within 5 sec; grade 3, falling off instantaneously.

Tail suspension test: The mouse was suspended by its tail and extension of hindlimbs observed (Garbuzova-Davis et al., J. Hematother. Stem Cell Res. 12:255-270, 2003). The deficits scores are: grade 0, normal; grade 1, partial hindlimb extension; grade 2, no hindlimb extension.

Data analysis: A total score of 18 from 5 different tests represents a complete loss of motor function, while a score of 0 means normal motor function. Results are quantified and expressed as mean±SEM. SPSS13.0 software is used to determine statistical significance using Log-Rank test for the onset and survival time of the disease. A p<0.05 is considered statistically significance.

Immunohistochemistry: Mice are anesthetized and perfused via the ascending aorta with pre-cooled phosphate-buffered saline (PBS, pH 7.4) for 1 min and then with pre-cooled 0.1 M PBS (pH 7.4) containing 4% paraformaldehyde for 15 min. The brain and lumbar spinal cord are removed, and post-fixed in the same fixative for 6 hr and then transferred to a solution of 0.1 M PBS containing 20% sucrose for 24 hr. A series of coronal sections of the fixed tissues are prepared in paraffin-embedded slides. The paraffin is removed from the tissue by xylene immersion. The endogenous peroxidase activity is blocked with 0.3% H₂O₂/methanol for 20 min. The sections are washed in 0.1 M PBS (pH 7.4), then boiled in 10 mM sodium citrate buffer (pH 6.0) for 20 min, followed by incubation overnight with primary antibody against phospho-GSK-3βSer9 (1:300, Abcam, Cambridge, Mass., USA). Biotinylated secondary antibody (Vector, Burlingame, Calif., USA) is then applied, and the immunohistochemical staining is revealed by the avidin-biotin complex using diaminobenzidine. The sections are finally dehydrated and covered.

### Tests in Alzheimer's Disease Model

AD is a progressive neurodegenerative disorder characterized by the deterioration of memory and higher cognitive functions. The neuropathological hallmarks of AD include age-related accumulation of Aβ peptides, neurofibrillary tangles, activation of glial cells in close vicinity to Aβ plaques associated with increased expression of several different cytokines and neuron loss in selective brain regions. Early-onset familial AD cases are caused by mutations in genes encoding amyloid precursor protein (APP) or presenilin 1 or 2 (PS1 and PS2) and are associated with increased production of the more amyloidogenic 42 amino acid long peptide, A1β1-42. The neurofibrillary tangles in AD are caused by abnormal production, processing and phosphorylation of the microtubule-associated protein Tau. There are multiple rodent models for AD, which trangenically express the APP either alone or together with PS1 or PS2 and or with variants of the Tau protein. One of these models is the Tg2576 transgenic mouse model of AD, which overexpresses a mutant form of amyloid precursor protein (APP), APP_{K670/671L}, linked to early onset familial AD, develops amyloid plaques and progressive cognitive deficits. In these mice, Aβ begins to rise rapidly at ˜6 months, coincident with the appearance of detergent-insoluble Aβ, and cognitive ability declines progressively thereafter Punctate, cored plaques are present in 7- to 8-month-old mice; mature, diffuse plaques appear at ˜12 months of age. Other AD rodent models include the triple transgenic mice expressing a mutant PS1 polypeptide, mutant human Tau protein and the human APP (U.S. Pat. No. 7,479,579), or double transgenic mice expressing a mutant human PS1 or 2 and the human APP (See Jüirgen Gotz & Lars M. Ittner, Nature Reviews Neuroscience 9: 532-544 (2008); and J Alzheimers Dis. 4:507-21 (2008)).

The transgenic mice are divided into treatment and control groups of 12-15 animals per group. Administration of drugs (antibody 5A5 or 3B4) or vehicle at 10 mg/kg is done i.p. once a week starting at the chosen age and continuing for up to 6 month.

The behavioral testing is performed at chosen ages. For cognition the mice are tested in Morris water maze and contextual fear conditioning tests. These tests measure both spatial and non-spatial memory as well as short- and long-term memory, and the performance in these tests is thought to be dependent on cortical structures such as hippocampus, neocortex and amygdala.

For pathology, the levels of soluble and insoluble Aβ peptides as well as the density of Aβ plaques are being measured in the brain and plasma

Contextual Fear Conditioning Test: The contextual fear conditioning test is described in Comery et al., J. Neurosci. 25(39):8898-902 (2005). The training and testing are conducted on two consecutive days, using a two-compartment (25×40×25 cm) Gemini operant chamber apparatus (San Diego Instruments) with stainless steel mouse grid floors. Training consists of placing a mouse in a chamber, illuminating stimulus and house lights on, and allowing exploration for 2 min. Afterward an auditory cue (conditioned stimulus (CS)) is presented for 15 s. A 2 s foot shock (1.5 mA; unconditioned stimulus) is administered for the final 2 s of the CS. This procedure is repeated, and the mouse is removed from the chamber 30 s later. 24 hours after training, the mouse is returned to the same chamber in which the training occurred (memory for context), and freezing behavior is recorded by the experimenter using time sampling (10 s intervals). Freezing is defined as lack of movement except that required for respiration. At the end of the 5 min context test, the mouse is returned to its home cage. One hour later, freezing is recorded in a novel environment (altered context) and in response to the cue (memory for cue). The novel environment consists of modifications including a white plastic divider diagonally bisecting the chamber, red plastic side walls and a black Plexiglas floor, and decreased illumination. The mouse is placed in the novel environment, and time sampling is used to score freezing for 3 min. The auditory cue (CS) is then presented for 3 min, and freezing is again scored. Freezing scores for each subject are expressed as a percentage for each portion of the test (memory for context, altered context, memory for cue).

Morris Water Maze Test: Water maze task was originally designed by Morris et al. (J Neurosci Methods. 1984; 11: 47-60). Testing is performed in a large dark-colored tank (120 cm in diameter) filled with clear water at a temperature of 25.0±1.0° C. A submerged platform (square platform: 15×15 cm; 1.5 cm below water surface) is placed in the middle of the one of the fours quadrants. The starting locations, which are labeled N, NE, E, SE, S, SW, W, NW, are located arbitrarily on the pool rim. The mice are lowered into the pool with their nose pointing toward the wall at one of the starting points. Mice with motor deficits and mice failing a simple visual discrimination task are eliminated. In the reference memory paradigm, the mice are given a series of 4 trials per day for 4 days. The platform is placed in the NW quadrant of the pool. The release point is changed in semi-random way between the training days. Each mouse is given a maximum of 60 s to find the submerged platform. Mice not finding the platform in 60 s are physically guided to it. Mice are allowed to stay on the platform for 20 s. On the fifth day, 24 hr after the last acquisition trial, the animals are tested in probe trial without the platform for 1 min. Before the reference memory task, the mice are trained with visible platform for 4 trials. The platform is made visible with 15 cm high pole with black and white stripes on it. Other trial parameters are same as during reference memory task. For each acquisition trial, the following parameters are analyzed: path length and escape latency to the platform (learning and memory functions), swim speed (motivation and motor functions), percent of swim time limited to outer annulus (15 cm) of the pool (thigmotaxic behaviour). During the probe trial, the time spent in target quadrant and target platform annulus (36-cm-diameter circular area surrounding platform), and crosses over the target platform position are measured (memory retention). The program also allows more detailed analysis of swim behaviour. The raw data is saved for possible further analysis.

Analysis of soluble and insoluble Aβ and of Aβ plaque: For terminal analysis of Aβ peptides in plasma, the mice are subjected to cardiac puncture and blood samples are collected into pre-cooled (ice bath) EDTA tubes. The tubes are kept on ice and plasma is separated by centrifugation at 2000 g (+4° C.) as soon as possible. 150-200 μl of plasma from each mouse is transferred into pre-cooled polypropylene tubes and kept frozen at −80° C. until testing by ELIZA.

For immunohistochemistry of Aβ plaque density, the brains are perfused with heparinized saline. Left hemisphere is post-fixed by immersion in 4% PFA in 0.1 M PB. After a brief wash with phosphate buffer, it is cryoprotected in 30% sucrose in PB for 2-3 days, after which it is frozen on liquid nitrogen and stored at −80° C. until immunohistochemistry is conducted. The other half of the brain (right) is fresh-frozen on liquid nitrogen and stored at −80° C. for analysis of insoluble A13 by ELISA.

Immunohistochemistry: Twenty-μm-thick coronal sections are prepared with a cryostat and mounted on SuperFrost Plus glass slides from the fixed, cryoprotected and frozen hemispheres. Selected sections are used for immunohistochemical analyses. Plaque load and the degree of amyloid aggregates in cortical and hippocampal structures are analyzed with amyloid beta immunohistochemical staining.

Amyloid Beta Immunohistochemistry: Briefly, tissue sections to be used in amyloid beta immunohistochemistry are thawed and air dried. The sections are reacted with an anti-amyloid beta-antibody (1:500; such as antibody from WO2 the Genetics Company, Switzerland) overnight at RT, or with Congo Red. Thereafter the sections are incubated with biotinylated secondary antibody and avidin-biotin complex (Vectastain Elite kit, Vector Laboratories, Burlingame, Calif.) for 2 h each, and peroxidase containing avidin-biotin complex is visualized using nickel-enhanced DAB as a substrate. Finally, the sections are rinsed, dehydrated, coverslipped and examined with a Leica 3000RBl microscope.

Image Analysis: Equally spaced coronal tissue sections along the antero-posterior axis of the hippocampus, 3-4 tissue sections from each animal, are analyzed for immunostaining intensity by ImagePro Plus software. Images of Aβ-immunoreactive staining are captured at defined light and filter settings in a brightfield-fluorescent microscope equipped with a cooled color CCD-camera. The captured images of Aβ-immunoreactive plaque deposits and intraneuronal Aβ aggregates are converted to grayscale images, processed with a delineation function to sharpen edges to allow an accurate segmentation. The images are segmented with an auto-threshold command (ImageProPlus, MediaCybernetics). The results are expressed as area fraction (stained areaₜₒₜ/measured areaₜₒₜ, expressed in %) and presented as mean±SEM among the tissue sections analyzed from each individual transgenic mouse.

Soluble and Insoluble Amyloid Beta 1-42 ELISA: The fresh-frozen hemisphere is used for biochemical analysis. Amyloid beta 1-42 ELISA analysis is applied to detect soluble and insoluble form of Aβ 1-42 in cortical and hippocampal structures. The brain tissue (ventral cortex and hippocampus) is homogenized and samples prepared according to the manufacturers detailed instructions (hAmyloid B42 Brain ELISA, The Genetics Company, Switzerland). Briefly the tissues are homogenized with a Dounce homogenizer (2×10 strokes on ice) in lysis buffer at a ratio of 1:10 (tissue weight:lysis buffer). Lysis buffer is Tris-buffered saline (TBS; 20 mM Tris-base and 137 mM NaCl, pH7.4) with protease inhibitors. The homogenate is centrifuged for 10 min at +4° C. with 13,000 rpm and the supernatant is divided in aliqouts and stored frozen at ˜20° C. prior to analyses (=Soluble A1β). An acid dissociation step may be added to remove therapeutic antibodies from Amyloid beta 1-42. The pellet is re-homogenized in cold 70% formic acid in distilled water, sonicated for 10 min, neutralized with 15× volume 1M Tris pH 7.4, and centrifuged for 10 min at +4° C. with 13,000 rpm. The supernatant is stored frozen at −20° C. (=Insoluble Aβ). Aβ 1-42 levels in serum as well as soluble and insoluble fractions of brain tissue samples are analyzed with ELISA using Amyloid Beta 1-42 ELISA kit (hAmyloid B42 Brain ELISA, The Genetics Company, Switzerland) according to instructions of the manufacturer. Standard curve range is from 25 to 500 pg/ml.

Statistical Analysis: All data are presented as mean±standard deviation (SD) or standard error of mean (SEM), and differences are considered to be statistically significant at the P<0.05 level. Statistical analysis is performed using StatsDirect statistical software. Differences among means are analyzed by using 1-way-ANOVA followed by an appropriate post hoc test. Within group comparison to the baseline is done by 2-way-ANOVA followed by an appropriate post hoc test. Non-parametric data is analyzed with Kruskal-Wallis ANOVA or Friedman ANOVA, respectively.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

All technical and scientific terms used herein have the same meaning as commonly understood to one of ordinary skill in the art to which this invention belongs unless clearly indicated otherwise.

### REFERENCES

Arber et al.; J. Cell Biol. 131:1083-1094 (1995).
Arikkath et al.; Current opinion in Neurobiology 13, 298-307 (2003).
Bacaj et al.; Science 322:744-747 (2008).
Bork et al.; Biochem. J. 279:908-910 (1991).
Beth et al.; Cell 131:1164 (2007).
Burns et al.; J. Comp Neurol. 500:11 (2007).
Bigio et al.; J. Neuropathol Exp Neurol. 60, 403 (2001).
Chafen et al.; J. Immunology 173: 3972-3978 (2004).
Chih et al.; Science 307, 1324-1328 (2005).
Christopherson et al.; Cell 120, 421-433 (2005).
Cole et al.; J. Compartive Neurology 491, 246-269 (2005).
Coleman et al.; Neurology, 63:1155-1162 (2004).
Colombatti et al.; MATRIX 13:297-306 (1993).
Davies et al.; Trends in Pharmacological Sciences 28:220-228 (2007).
Dickman et al.; J Neurosci 28, 31-38 (2008).
Davis; Neuron 26, 551-554 (2000).
DeKosky; Annals of Neurology, 27:457 (1990).
Edwards et al.; FEBS LETT. 358: 283-286 (1995).
Eroglu et al.; Cell 139, 380-392 (2009).
Field et al.; Proc. Natl. Acad. Sci. U.S.C. 103, 17537-17542 (2006).
Frey et al.; The Journal of Neuroscience 20:2534 (2000).
Ferri et al.; Current Biology 13, 669 (2003).
Garner et al.; Cell and Tissue Research 326, 249-262 (2006).
Gee et al.; J. Biol. Chem. 271, 5768-5776 (1996).
Jeffrey et al.; Neuropathology & Applied Neurobiology 26(14), pp. 41-54 (2000).
Huang et al.; J. Biol. Chem. 270:19008 (1995).
Huang et al.; Domain localization and correlation with inhibition of function of workshop CD11a mAb. S. F. Schlossman, and L. Boumsell, and W. Gilks, and J. Harlan, and T. Kishimoto, and T. Morimoto, and J. Ritz, and S. Shaw, and R. Silverstein, and T. Springer, and T. Tedder, and R. Todd, eds. Leucocyte Typing V: White Cell Differentiation Antigens 1595. Oxford Univ. Press, New York (1995).
Huang et al.; J. Biol. Chem. 275:21514 (2000).
Hen et al., Journal of Neurochemistry 105, 78 (2008).
Kennedy et al. Annu Rev Neurosci. 29:325-62 (2006).
Klugbauer et al.; Journal of Bioenergetics and Biomembranes 35, 639-647 (2003).
Landis et al.; J. Cell Biol. 120:1519 (1993)
Lee et al.; Cell 80:631-638 (1995).
Liauw et al., J. Cereb. Blood Flow Metab. 28:1722-1732, 2008.
Zhao et al., Nature Neuroscience 9, 234-242 (2006).
Mack et al., Journal of Neuroscience Research. 83, 5883 (2006).
Marais et al., Mol. Pharmacol. 59(5):1243-8 (2001).
Nishimune et al.; Nature 432, 580-587 (2004).
Perkins et al.; J. Mol. Biol. 238:104-119 (1994).
Qu et al.; Proc. Natl. Acad. Sci. U.S.A. 92: 10277-10281 (1995).
Ruggeri et al.; FASEB J. 7: 308-316 (1993).
Scheiffele et al.; Annu Rev Neurosci 26, 485-508 (2003).
Susman, (2007). Society for neuroscience abstract, Identification of the Thrombospondin receptor that promotes CNS synaptogenesis, 571.2/G52.
Shoichi et al.; Acta Neuropathologica 88, 222 (1994).
Taylor et al.; Neuroscience. 155(2):510-21 (2008).
Umemori et al.; Cell 118, 257-270 (2004).
Washbourne et al.; J Neurosci 24, 9244-9249 (2004).
Wilkins et al.; J. Biol. Chem. 271, pp. 3046-3051 (1996).
Whittaker et al.; Molecular Biology of the Cell 13, 3369-3387 (2002).
Wycisk et al.; Investigative Ophthalmology & Visual Science 47, 3523-3530 (2006).
Yoshiyama et al., Neuron 53:337 (2007).
Ziv et al.; Nature Reviews 5, 385-399 (2004).
Xu et al.; Thrombospondin 1 accelerates synaptogenesis in hippocampal neurons through neuroligin 1; Nature Neuroscience, published online Nov. 15, 2009.

1. An isolated antibody that specifically binds to an epitope in a von Willebrand Factor A (VWF-A) domain of a calcium channel subunit selected from the group consisting of α2δ1, α2δ2, α2δ3, and α2δ4 and induces synaptogenesis of a neuronal cell expressing the calcium channel subunit.

2. The antibody of claim 1, wherein the antibody binds to an epitope within amino acids of about 253 to about 430 of SEQ ID NO:1.

3. The antibody of claim 1, wherein the antibody binds to an epitope within amino acids of about 386 to about 425 of SEQ ID NO:1.

4. The antibody of claim 2, wherein the antibody binds to a peptide having the amino acid sequence of SEQ ID NO:6 (MZp110).

5. The antibody of claim 1, wherein the antibody binds to an epitope within amino acids of about 291 to about 469 of SEQ ID NO:2.

6. The antibody of claim 1, wherein the antibody binds to an epitope within amino acids of about 421 to about 464 of SEQ ID NO:2.

7. The antibody of claim 5, wherein the antibody binds to a peptide having the amino acid sequence of SEQ ID NO:5 (MZp109).

8. The antibody of claim 1, wherein the antibody binds to an epitope within amino acids of about 256 to about 438 of SEQ ID NO:3.

9. The antibody of claim 1, wherein the antibody binds to an epitope within amino acids of about 291 to about 473 of SEQ ID NO:4.

10. The antibody of claim 1, wherein the antibody is a monoclonal antibody.

11. The antibody of claim 1, wherein the antibody is a human antibody, a humanized antibody or a chimeric antibody.

12. The antibody of claim 1, which is antibody 5A5 or an antigen-binding fragment thereof.

13. The antibody of claim 1, which comprises the three CDRs from the heavy chain of antibody 5A5, and/or the three CDRs from the light chain of antibody 5A5.

14. The antibody of claim 1, which comprises the heavy chain variable region sequence of antibody 5A5, and/or the light chain of variable region sequence of antibody 5A5.

15. The antibody of claim 1, which is antibody 3B4 or an antigen-binding fragment thereof.

16. The antibody of claim 1, which comprises the three CDRs from the heavy chain of antibody 3B4, and/or the three CDRs from the light chain of antibody 3B4.

17. The antibody of claim 1, which comprises the heavy chain variable region sequence of antibody 3B4, and/or the light chain of variable region sequence of antibody 3B4.

18. An isolated polynucleotide comprising a nucleotide sequence encoding the antibody of claim 1.

19. A vector comprising the polynucleotide of claim 18.

20. The vector of claim 19, which is an expression vector comprising the polynucleotide operably linked to an expression control sequence.

21. A host cell comprising the polynucleotide of claim 18.

22. A method for producing an antibody of claim 1 comprising culturing a cell that produces the antibody and recovering the antibody from the cell culture.

23. A method of inducing synaptogenesis in an individual comprising administering to the individual in need of synaptogenesis an effective dose of an antibody of claim 1.

24. The method of claim 23, wherein the individual has suffered synapse loss as a result of senescence.

25. The method of claim 23, wherein the individual has suffered synapse loss as a result of Alzheimer's disease, Parkinson's disease, ALS, multiple sclerosis, Huntington disease, Down syndrome, spinal mascular atrophy, stroke, spinal cord injury, myofiber atrophy, denervation atrophy, or glaucoma.

26. The method of claim 23, wherein the individual has suffered a macular degeneration, a hearing loss, a diabetic neuropathy, or a chemotherapy induced neuropathy.

27. The method of claim 23, wherein the individual has suffered synapse loss as a result of a psychiatric disorder selected from the group consisting of depression, schizophrenia, autism, and aggression.

28. The method of claim 23, wherein the individual has suffered synapse loss as a result of a viral infection selected from the group consisting of poliomyelitis, west Nile virus, or HIV.

29. The method of claim 23, wherein the individual has suffered synapse loss as a result of a Prion disease.

30. The method of claim 29, wherein said Prion disease is Creutzfeldt-Jakob disease.

31. The method of claim 23, wherein said synaptogenesis is increased at a neuromuscular junction.

32. The method of claim 23, wherein said synaptogenesis is increased in a sense organ.

33. A method for inducing neurite outgrowth in an individual comprising administering to the individual in need of neurite outgrowth an effective dose of an antibody of claim 1.
